(19) United States
(12) Patent Application Publication
(10) Pub. No.: US 2022/0041582 A1
(43) Pub. Date: Feb. 10, 2022

(54) ISOXAZOLINE COMPOUNDS FOR CONTROLLING INVERTEBRATE PESTS

(71) Applicant: FMC Corporation, Philadelphia, PA (US)

(72) Inventors: Ming XU, Newark, DE (US); George Philip LAHM, Wilmington, DE (US)

(21) Appl. No.: 17/276,064

(22) PCT Filed: Sep. 11, 2019
(86) PCT No.: PCT/US19/50534

§ 371 Date: Mar. 12, 2021

Related U.S. Application Data
(60) Provisional application No. 62/730,067, filed on Sep. 12, 2018.

(51) Int. Cl. C07D 413/08; A01N 43/80; C07D 413/14

(52) U.S. Cl. CPC C07D 413/08; C07D 413/14; A01N 43/80

(57) ABSTRACT

Disclosed are compounds of Formula 1, wherein R¹, R², R³ and J are as defined in the disclosure. Also disclosed are compositions containing the compounds of Formula 1 and methods for controlling an invertebrate pest comprising contacting the invertebrate pest or its environment with a biologically effective amount of a compound or a composition of the disclosure.

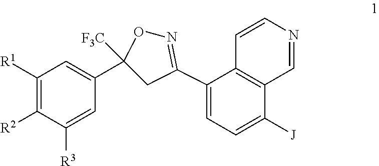

### FIELD

This disclosure relates to certain isoxazoline compounds and compositions suitable for agronomic and nonagronomic uses, and methods of their use for controlling invertebrate pests such as arthropods in both agronomic and nonagronomic environments.

### BACKGROUND

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, household, turf, wood products, and public health is also important. Many products are commercially available for these purposes, but the need continues for new compounds that are more effective, less costly, less toxic, environmentally safer or have different sites of action.

### SUMMARY

This disclosure is directed to compounds of Formula 1 (including all stereoisomers, enantiomers, or diastereomers thereof), compositions containing them, and their use for controlling invertebrate pests:

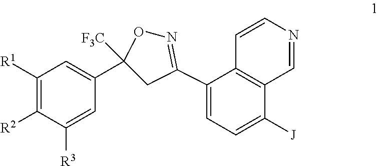

wherein
J is

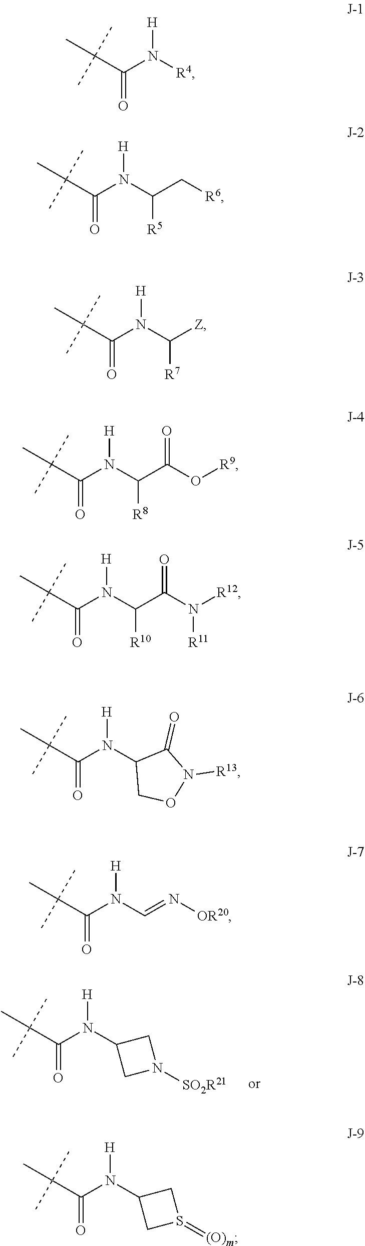

R¹ is H, Cl or CF₃;
R² is H, F or Cl;
R³ is H, Cl or CF₃;
R⁴ is C₁-C₆ alkyl, C₂-C₆ alkenyl, C₂-C₆ alkynyl, C₃-C₆ cycloalkyl, C₃-C₆ cycloalkenyl, C₄-C₈ alkylcycloalkyl or C₄-C₈ cycloalkylalkyl, each unsubstituted or substituted with substituents independently selected from halogen, cyano and CO₂R¹⁸;
R⁵ is H or C₁-C₄ alkyl;
R⁶ is OR¹⁴ or S(O)ₙR¹⁵;
R⁷ is H or C₁-C₄ alkyl;
R⁸ is H or C₁-C₄ alkyl;
R⁹ is H; or C₁-C₄ alkyl, unsubstituted or substituted with substituents independently selected from halogen, cyano, OR¹⁶, S(O)ₙR¹⁷ and CO₂R¹⁸;
R¹⁰ is H or C₁-C₄ alkyl;
R¹¹ is H or C₁-C₄ alkyl;
R¹² is H; or C₁-C₄ alkyl, unsubstituted or substituted with substituents independently selected from halogen, cyano, OR¹⁶, S(O)₁R¹⁷ and CO₂R¹⁸;
R¹³ is H, C₁-C₄ alkyl or C₁-C₄ haloalkyl;
R¹⁴ is C₁-C₄ alkyl;
R¹⁵ is H, C₁-C₄ alkyl or C₁-C₄ haloalkyl;
each R¹⁶ is independently C₁-C₄ alkyl or C₁-C₄ haloalkyl;
each R¹⁷ is independently C₁-C₄ alkyl or C₁-C₄ haloalkyl;
each R¹⁸ is independently C₁-C₄ alkyl or C₁-C₄ haloalkyl;
Z is pyridinyl, pyrimidinyl, pyrazinyl, pyridazinyl or tetrahydrofuranyl, each unsubstituted or substituted with R¹⁹;
each R¹⁹ is independently halogen, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂-C₅ alkoxycarbonyl, C₂-C₅ alkylaminocarbonyl and C₃-C₅ dialkylaminocarbonyl;
R²⁰ is H, C₁-C₄ alkyl or C₁-C₄ haloalkyl;
R²¹ is fluoro, C₁-C₄ alkyl, C₁-C₄ haloalkyl, C₁-C₆ alkoxy, C₁-C₆ haloalkoxy, amino or C₁-C₆ alkylamino;
m is 0, 1 or 2; and
each n is independently 0, 1 or 2;
provided that
(i) when J is J-1, R¹ is Cl, R² is H and R³ is Cl, then R⁴ is other than —CH₂CH₃, —CH₂CF₃ or —CH₂(cyclopropyl);
(ii) when J is J-3, R¹ is Cl, R² is H, R³ is Cl and R⁷ is H, then Z is other than 2-pyridinyl; and
(iii) when J is J-5, R¹ is Cl, R² is H, R³ is Cl, and R¹⁰ and R¹¹ are H, then R¹² is other than —CH₂CF₃.
(iv) when R¹ is H, then R³ is other than H; and when R³ is H, then R¹ is other than H.

This disclosure also provides compounds of Formula 1. In one embodiment this disclosure provides a composition comprising a compound of Formula 1 and at least one additional component selected from the group consisting of surfactants, solid diluents and liquid diluents. In one embodiment, this disclosure also provides a composition for controlling an invertebrate pest comprising a compound of Formula 1 and at least one additional component selected from the group consisting of surfactants, solid diluents and liquid diluents, said composition optionally further comprising at least one additional biologically active compound or agent.

This disclosure provides a method for controlling an invertebrate pest comprising contacting the invertebrate pest or its environment with a biologically effective amount of a compound of Formula 1 and compositions thereof. This disclosure also relates to such method wherein the invertebrate pest or its environment is contacted with a composition comprising a biologically effective amount of a compound of Formula 1, and at least one additional component selected from the group consisting of surfactants, solid diluents and liquid diluents, said composition optionally further comprising a biologically effective amount of at least one additional biologically active compound or agent.

This disclosure also provides a method for protecting a seed from an invertebrate pest comprising contacting the seed with a biologically effective amount of a compound of Formula 1 and compositions comprising a Compound of Formula 1. This disclosure also relates to the treated seed.

This disclosure also provides a method for increasing vigor of a crop plant comprising contacting the crop plant, the seed from which the crop plant is grown or the locus (e.g., growth medium) of the crop plant with a biologically effective amount of a compound of Formula 1 or compositions comprising a compound of Formula 1.

### DETAILED DESCRIPTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the embodiments. The term “consisting essentially of” occupies a middle ground between “comprising” and “consisting of”.

Where applicants have defined an embodiment or a portion thereof with an open-ended term such as “comprising,” it should be readily understood that (unless otherwise stated) the description should be interpreted to also describe such an embodiment using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the disclosure are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods, nematodes and helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes members of the phylum Nematoda.

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, and public health applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) an invertebrate pest to be controlled or its environment, or to a plant, the seed from which the plant is grown, or the locus of the plant (e.g., growth medium) to protect the plant from injury by the invertebrate pest or for other desired effect (e.g., increasing plant vigor).

It also is understood that any numerical range recited herein includes all values from the lower value to the upper value. For example, if a weight ratio range is stated as 1:50, it is intended that values such as 2:40, 10:30, or 1:3, etc., are expressly enumerated in this specification. These are only examples of what is specifically intended, and all possible combinations of numerical values between and including the lowest value and the highest value enumerated are to be considered to be expressly stated in this application.

One skilled in the art can easily determine through simple experimentation the biologically effective amounts of active ingredients necessary for the desired spectrum of biological activity. It will be evident that including these additional components can expand the spectrum of invertebrate pests controlled beyond the spectrum controlled by the compound of Formula 1 alone.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers.

“Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—.

The chemical abbreviations S(O) and S(═O) as used herein represent a sulfinyl moiety. The chemical abbreviations SO₂, S(O)₂ and S(═O)₂ as used herein represent a sulfonyl moiety. The chemical abbreviations C(O) and C(═O) as used herein represent a carbonyl moiety. The chemical abbreviations CO₂, C(O)O and C(═O)O as used herein represent an oxycarbonyl moiety.

The total number of carbon atoms in a substituent group is indicated by the “Cᵢ-Cⱼ” prefix. For example, C₁-C₄ alkyl designates methyl, ethyl, and the various propyl and butyl isomers.

When a compound is substituted with a substituent bearing a subscript that indicates the number of said substituents can exceed 1, said substituents (when they exceed 1) are independently selected from the group of defined substituents. Further, when the subscript indicates a range, e.g. (R)ᵢ₋ⱼ, then the number of substituents may be selected from the integers between i and j inclusive. When a group contains a substituent which can be hydrogen, then when this substituent is taken as hydrogen, it is recognized that this is equivalent to said group being unsubstituted. When one or more positions on a group are said to be “not substituted” or “unsubstituted”, then hydrogen atoms are attached to take up any free valency.

Unless otherwise indicated, heterocyclic rings and ring systems can be attached through any available carbon or nitrogen by replacement of a hydrogen on said carbon or nitrogen.

When a substituent is a 5- or 6-membered nitrogen-containing heterocyclic ring, it may be attached to the remainder of Formula 1 though any available carbon or nitrogen ring atom, unless otherwise described.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this disclosure can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

Compounds selected from Formula 1 typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. Compounds of this disclosure may exist as one or more crystalline polymorphs. This disclosure comprises both individual polymorphs and mixtures of polymorphs, including mixtures enriched in one polymorph relative to others. For a comprehensive discussion of polymorphism see R. Hilfiker, Ed., Polymorphism in the Pharmaceutical Industry, Wiley-VCH, Weinheim, 2006.

Embodiments of the present disclosure as described in the Summary include those described below. In the following Embodiments, reference to “a compound of Formula 1” includes the definitions of substituents specified in the Summary unless further defined in the Embodiments.
Embodiment 1a. A compound of Formula 1 wherein R¹ is CF₃ or H, R² is F or H, and R³ is H or CF₃.
Embodiment 1b. A compound of Formula 1 wherein R¹ is Cl or CF₃, R² is H or F, and R³ is Cl.
Embodiment 1c. A compound of Formula 1 wherein R¹ is Cl or H, R² is F or H, and R³ is CF₃.
Embodiment 1d. A compound of Formula 1 wherein R¹ is CF₃ or H, R² is F or H, and R³ is H or CF₃.
Embodiment 1e. A compound of Formula 1 wherein R¹ is Cl or CF₃, R² is F, and R³ is Cl.
Embodiment 1f. A compound of Formula 1 wherein R¹ is Cl or H, R₂ is F or H, and R³ is CF₃.
Embodiment 1g. A compound of Formula 1 wherein R¹ is Cl, R² is H, and R³ is Cl.
Embodiment 1h. A compound of Formula 1 wherein R¹ is Cl, R² is F, and R³ is Cl.
Embodiment 1i. A compound of Formula 1 wherein R¹ is H, R² is F, and R³ is Cl.
Embodiment 1j. A compound of Formula 1 wherein R¹ is H, R² is F, and R³ is CF₃.
Embodiment 1k. A compound of Formula 1 wherein R¹ is H, R² is H, and R³ is CF₃.
Embodiment 1l. A compound of Formula 1 wherein R¹ is Cl, R² is H, and R³ is CF₃.
Embodiment 1m. A compound of Formula 1 wherein R¹ is CF₃, R² is H, and R³ is H.
Embodiment 1n. A compound of Formula 1 wherein R¹ is CF₃, R² is F, and R³ is H.
Embodiment 1o. A compound of Formula 1 wherein R¹ is Cl, R² is F, and R³ is CF₃.
Embodiment 2a. A compound of Formula 1 wherein J is J-1.
Embodiment 2b. A compound of Embodiment 2a wherein R⁴ is C₁-C₆ alkyl.
Embodiment 2c. A compound of Embodiment 2a wherein R⁴ is C₂-C₆ alkenyl.
Embodiment 2d. A compound of Embodiment 2a wherein R⁴ is C₂-C₆ alkynyl.
Embodiment 2e. A compound of Embodiment 2a wherein R⁴ is C₃-C₆ cycloalkyl.
Embodiment 2f A compound of Embodiment 2a wherein R⁴ is C₄-C₈ alkylcycloalkyl.
Embodiment 2g. A compound of Embodiment 2f wherein R⁴ is CH₂-c-Pr.
Embodiment 2h. A compound of any one of Embodiments 2a-2g wherein R⁴ is substituted with substituents independently selected from halogen, cyano and CO₂R¹⁸.
Embodiment 2i. A compound of Embodiment 2h wherein the substituent is halogen.
Embodiment 2j. A compound of Embodiment 2i wherein the halogen is F.
Embodiment 2k. A compound of Embodiment 2h wherein the substituent is cyano.
Embodiment 2l. A compound of Embodiment 2h wherein the substituent is CO₂R¹⁸.
Embodiment 2 m. A compound of Embodiment 2l wherein R¹⁸ is C₁-C₄ alkyl.
Embodiment 3a. A compound of Formula 1 wherein J is J-2.
Embodiment 3b. A compound of Embodiment 3a wherein R⁵ is H or Me.
Embodiment 3c. A compound of Embodiment 3a wherein R⁶ is OMe, SMe, S(O)Me or SO₂Me.
Embodiment 3d. A compound of Embodiment 3a wherein R⁵ is H or Me, and R⁶ is OMe, SMe, S(O)Me or SO₂Me.
Embodiment 4a. A compound of Formula 1 wherein J is J-3.
Embodiment 4b. A compound of Embodiment 4a wherein R⁷ is H or Me.
Embodiment 4c. A compound of Embodiment 4a wherein Z is 2-pyridinyl or 2-pyrimidinyl.
Embodiment 4d. A compound of Embodiment 4a wherein R⁷ is H or Me, and Z is 2-pyridinyl or 2-pyrimidinyl.
Embodiment 4e. A compound of Embodiment 4a wherein R⁷ is H or Me, and Z is 2-pyrimidinyl.
Embodiment 5a. A compound of Formula 1 wherein J is J-4.
Embodiment 5b. A compound of Embodiment 5a wherein R⁸ is H or Me.
Embodiment 5c. A compound of Embodiment 5a wherein R⁹ is H or C₁-C₃ alkyl.
Embodiment 5d. A compound of Embodiment 5a wherein R⁸ is H or Me, and R⁹ is H or C₁-C₃ alkyl.
Embodiment 6a. A compound of Formula 1 wherein J is J-5.
Embodiment 6b. A compound of Embodiment 6a wherein R¹¹ is H.
Embodiment 6c. A compound of Embodiment 6b wherein R¹⁰ is H or Me.
Embodiment 6d. A compound of Embodiment 6b wherein R¹² is H, C₁-C₂ alkyl, CH₂CF₃ or CH₂CN.
Embodiment 6e. A compound of Embodiment 6b wherein R¹⁰ is H or Me, and R¹² is H, C₁-C₂ alkyl, CH₂CF₃ or CH₂CN.
Embodiment 7a. A compound of Formula 1 wherein J is J-6.
Embodiment 7b. A compound of Embodiment 7a wherein R¹³ is H, C₁-C₂ alkyl or CH₂CF₃.
Embodiment 8a. A compound of Formula 1 wherein J is J-7.
Embodiment 8b. A compound of Embodiment 8a wherein R²⁰ is methyl.
Embodiment 9a. A compound of Formula 1 wherein J is J-8.
Embodiment 9b. A compound of Embodiment 9a wherein R²¹ is fluoro, C₁-C₃ alkyl, cyclopropyl or dimethylamino.
Embodiment 10a. A compound of Formula 1 wherein J is J-9.
Embodiment 10b. A compound of Embodiment 10a wherein m is 1.
Embodiment 11a. A compound of Formula 1 wherein R¹ is Cl, H, or CF₃, R² is H, or F, R³ is H, Cl, or CF₃ and J is J-1, J-2, or J-3.
Embodiment 11b. A compound of Embodiment 11a wherein R¹ is Cl, or H.
Embodiment 11c. A compound of Embodiment 11a or 11b wherein R² is H.
Embodiment 11d. A compound of any one of Embodiments 11a-c wherein R³ is Cl, or CF₃.
Embodiment 11e. A compound of any one of Embodiments 11a-d wherein R³ is Cl.
Embodiment 11f. A compound of any one of Embodiments 11a-e wherein J is J-1, or J-3
Embodiment 11g. A compound of any one of Embodiments 11a-f wherein J is J-1.
Embodiment 11h. A compound of any one of Embodiments 11a-g wherein J is J-1 and J-1 is —C(O)NH(t-Bu), —C(O)NHCH₂CH₃, —C(O)NHCH₂CCH, —C(O)NH(c-Pr), or —C(O)NHCH₂(c-Pr).
Embodiment 11i. A compound of Embodiment 11d wherein J-1 is C(O)NH(c-Pr), or —C(O)NHCH₂(c-Pr).

Embodiments of this disclosure, including Embodiments 1-11i above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this disclosure, including Embodiments 1-11i above as well as any other embodiments described herein, and any combination thereof, pertain to the compositions and methods of the present disclosure.

Specific embodiments include compounds of Formula 1 selected from the group consisting of compounds 5-[5-(3,5-dichloro-4-fluorophenyl)-4,5-dihydro-5-(trifluoromethyl)-3-isoxazolyl]-N-(1,1-dimethylethyl)-8-isoquinolinecarboxamide (Compound 2), N-(cyclopropylmethyl)-5-[5-(3,5-dichloro-4-fluorophenyl)-4,5-dihydro-5-(trifluoromethyl)-3-isoxazolyl]-8-isoquinolinecarboxamide (Compound 3), 5-[5-(3,5-dichloro-4-fluorophenyl)-4,5-dihydro-5-(trifluoromethyl)-3-isoxazolyl]-N-(2-pyrimidinylmethyl)-8-isoquinolinecarboxamide (Compound 4), N-cyclopropyl-5-[5-(3,5-dichloro-4-fluorophenyl)-4,5-dihydro-5-(trifluoromethyl)-3-isoxazolyl]-8-isoquinolinecarboxamide (Compound 23), 5-[5-[3-chloro-5-(trifluoromethyl)phenyl]-4,5-dihydro-5-(trifluoromethyl)-3-isoxazolyl]-N-(cyclopropylmethyl)-8-isoquinolinecarboxamide (Compound 36), N-cyclopropyl-5-[4,5-dihydro-5-(trifluoromethyl)-5-[3-(trifluoromethyl)phenyl]-3-isoxazolyl]-8-isoquinolinecarboxamide (Compound 16), N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 17), N-(cyclopropylmethyl)-5-[5-[4-fluoro-3-(trifluoromethyl)phenyl]-4,5-dihydro-5-(trifluoromethyl)-3-isoxazolyl]-8-isoquinolinecarboxamide (Compound 58), 5-[5-[3-chloro-5-(trifluoromethyl)phenyl]-4,5-dihydro-5-(trifluoromethyl)-3-isoxazolyl]-N-cyclopropyl-8-isoquinolinecarboxamide (Compound 35), 5-[5-[4-fluoro-3-(trifluoromethyl)phenyl]-4,5-dihydro-5-(trifluoromethyl)-3-isoxazolyl]-N-2-propyn-1-yl-8-isoquinolinecarboxamide (Compound 74), 5-[5-(3,5-dichloro-4-fluorophenyl)-4,5-dihydro-5-(trifluoromethyl)-3-isoxazolyl]-N-ethyl-8 soquinolinecarboxamide (Compound 46), 5-[5-(3,5-dichloro-4-fluorophenyl)-4,5-dihydro-5-(trifluoromethyl)-3-isoxazolyl]-N-2-propyn-1-yl-8-isoquinolinecarboxamide (Compound 48), N-cyclopropyl-5-[5-(3,5-dichlorophenyl)-4,5-dihydro-5-(trifluoromethyl)-3-isoxazolyl]-8-isoquinolinecarboxamide (Compound 70), N-cyclopropyl-5-[5-[4-fluoro-3-(trifluoromethyl)phenyl]-4,5-dihydro-5-(trifluoromethyl)-3-isoxazolyl]-8-isoquinolinecarboxamide (Compound 55), S—N-cyclopropyl-5-[5-(3,5-dichloro-4-fluorophenyl)-4,5-dihydro-5-(trifluoromethyl)-3-isoxazolyl]-8-isoquinolinecarboxamide (Compound 82) and S—N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 99).

In one embodiment, the compound of Formula 1 is selected from N-cyclopropyl-5-[4,5-dihydro-5-(trifluoromethyl)-5-[3-(trifluoromethyl)phenyl]-3-isoxazolyl]-8-isoquinolinecarboxamide (Compound 16), N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 17), N-cyclopropyl-5-[5-(3,5-dichlorophenyl)-4,5-dihydro-5-(trifluoromethyl)-3-isoxazolyl]-8-isoquinolinecarboxamide (Compound 70), N-cyclopropyl-5-[5-[4-fluoro-3-(trifluoromethyl)phenyl]-4,5-dihydro-5-(trifluoromethyl)-3-isoxazolyl]-8-isoquinolinecarboxamide (Compound 55), S—N-cyclopropyl-5-[5-(3,5-dichloro-4-fluorophenyl)-4,5-dihydro-5-(trifluoromethyl)-3-isoxazolyl]-8-isoquinolinecarboxamide (Compound 82), and S—N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 99).

In one embodiment, the compounds of Formula 1 are those wherein the compound is selected from the compounds in Table 1.

TABLE 1

| Compound | Compound Structure | Chemical name |
|---|---|---|
| 2 | 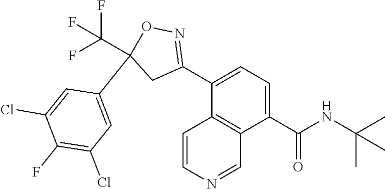 [SMILES: CC(C)(C)NC(=O)c1ccc(C2=NOC(c3cc(Cl)c(F)c(Cl)c3)(C(F)(F)F)C2)c2ccncc12] | 5-[5-(3,5-dichloro-4- fluorophenyl)-4,5- dihydro-5- (trifluoromethyl)-3- isoxazolyl]-N-(1,1- dimethylethyl)-8- isoquinoline- carboxamide |
| 3 | 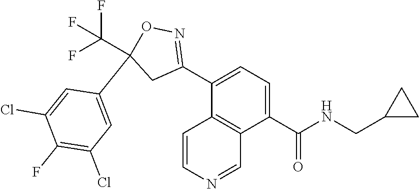 [SMILES: O=C(NCC1CC1)c1ccc(C2=NOC(c3cc(Cl)c(F)c(Cl)c3)(C(F)(F)F)C2)c2ccncc12] | N- (cyclopropylmethyl)-5- [5-(3,5-dichloro-4- fluorophenyl)-4,5- dihydro-5- (trifluoromethyl)-3- isoxazolyl]-8- isoquinoline- carboxamide |
| 4 | 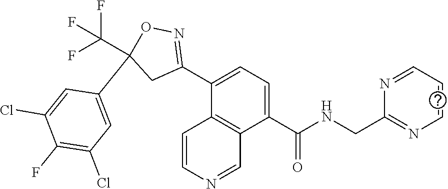 [SMILES: O=C(NCc1ncccn1)c1ccc(C2=NOC(c3cc(Cl)c(F)c(Cl)c3)(C(F)(F)F)C2)c2ccncc12] | 5-[5-(3,5-dichloro-4- fluorophenyl)-4,5- dihydro-5- (trifluoromethyl)-3- isoxazolyl]-N-(2- pyrimidinylmethyl)-8- isoquinoline- carboxamide |
| 23 | 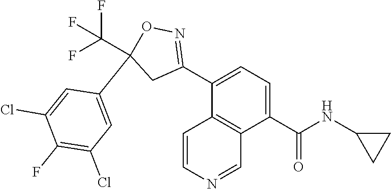 [SMILES: O=C(NC1CC1)c1ccc(C2=NOC(c3cc(Cl)c(F)c(Cl)c3)(C(F)(F)F)C2)c2ccncc12] | N-cyclopropyl-5-[5- (3,5-dichloro-4- fluorophenyl)-4,5- dihydro-5- (trifluoromethyl)-3- isoxazolyl]-8- isoquinoline- carboxamide |
| 36 | 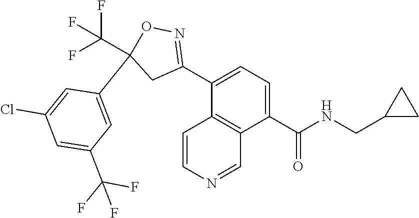 [SMILES: O=C(NCC1CC1)c1ccc(C2=NOC(c3cc(Cl)cc(C(F)(F)F)c3)(C(F)(F)F)C2)c2ccncc12] | 5-[5-[3-chloro-5- (trifluoromethyl)phenyl]- 4,5-dihydro-5- (trifluoromethyl)-3- isoxazolyl]-N- (cyclopropylmethyl)-8- isoquinoline- carboxamide |
| 16 | 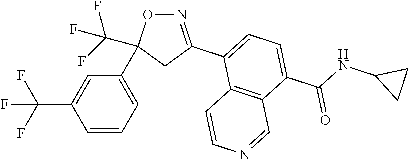 [SMILES: O=C(NC1CC1)c1ccc(C2=NOC(c3cccc(C(F)(F)F)c3)(C(F)(F)F)C2)c2ccncc12] | N-cyclopropyl-5-[4,5- dihydro-5- (trifluoromethyl)-5-[3- (trifluoromethyl)phenyl]- 3-isoxazolyl]-8- isoquinoline- carboxamide |
| 17 | 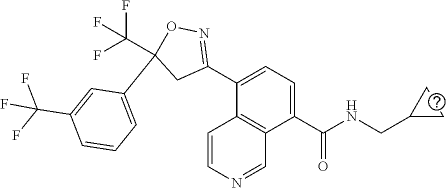 [SMILES: O=C(NCC1CC1)c1ccc(C2=NOC(c3cccc(C(F)(F)F)c3)(C(F)(F)F)C2)c2ccncc12] | N- (cyclopropylmethyl)-5- [4,5-dihydro-5- (trifluoromethyl)-5-[3- (trifluoromethyl)phenyl]- 3-isoxazolyl]-8- isoquinoline- carboxamide |
| 58 | 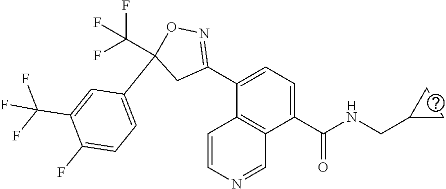 [SMILES: O=C(NCC1CC1)c1ccc(C2=NOC(c3ccc(F)c(C(F)(F)F)c3)(C(F)(F)F)C2)c2ccncc12] | N- (cyclopropylmethyl)-5- [5-[4-fluoro-3- (trifluoromethyl)phenyl]- 4,5-dihydro-5- (trifluoromethyl)-3- isoxazolyl]-8- isoquinoline- carboxamide |
| 35 | 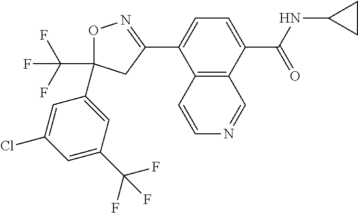 [SMILES: O=C(NC1CC1)c1ccc(C2=NOC(c3cc(Cl)cc(C(F)(F)F)c3)(C(F)(F)F)C2)c2ccncc12] | 5-[5-[3-chloro- 5- (trifluoromethyl)phenyl]- 4,5-dihydro-5- (trifluoromethyl)-3- isoxazolyl]-N- cyclopropyl-8- isoquinoline- carboxamide |
| 74 | 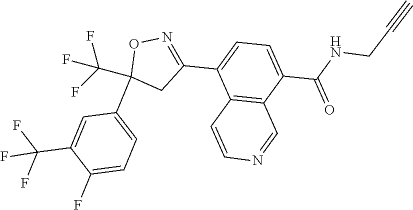 [SMILES: C#CCNC(=O)c1ccc(C2=NOC(c3ccc(F)c(C(F)(F)F)c3)(C(F)(F)F)C2)c2ccncc12] | 5-[5-[4-fluoro- 3- (trifluoromethyl)phenyl]- 4,5-dihydro-5- (trifluoromethyl)-3- isoxazolyl]-N-2- propyn-1-yl-8- isoquinoline- carboxamide |
| 46 | 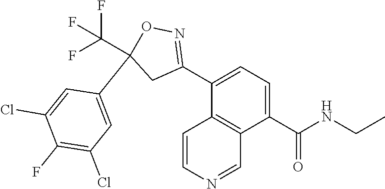 [SMILES: CCNC(=O)c1ccc(C2=NOC(c3cc(Cl)c(F)c(Cl)c3)(C(F)(F)F)C2)c2ccncc12] | 5-[5-(3,5- dichloro-4- fluorophenyl)-4,5- dihydro-5- (trifluoromethyl)-3- isoxazolyl]-N-ethyl-8- isoquinoline- carboxamide (Compound 46) |
| 48 | 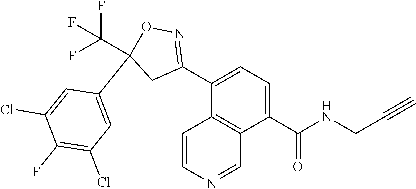 [SMILES: C#CCNC(=O)c1ccc(C2=NOC(c3cc(Cl)c(F)c(Cl)c3)(C(F)(F)F)C2)c2ccncc12] | 5-[5-(3,5- dichloro-4- fluorophenyl)-4,5- dihydro-5- (trifluoromethyl)-3- isoxazolyl]-N-2- propyn-1-yl-8- isoquinoline- carboxamide |
| 70 | 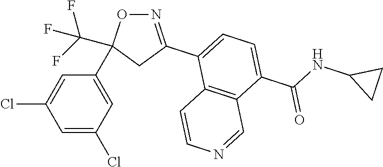 [SMILES: O=C(NC1CC1)c1ccc(C2=NOC(c3cc(Cl)cc(Cl)c3)(C(F)(F)F)C2)c2ccncc12] | N-cyclopropyl- 5-[5-(3,5- dichlorophenyl)-4,5- dihydro-5- (trifluoromethyl)-3- isoxazolyl]-8- isoquinoline- carboxamide |
| 55 | 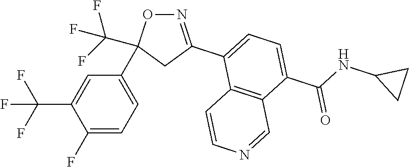 [SMILES: O=C(NC1CC1)c1ccc(C2=NOC(c3ccc(F)c(C(F)(F)F)c3)(C(F)(F)F)C2)c2ccncc12] | N-cyclopropyl- 5-[5-[4-fluoro-3- (trifluoromethyl)phenyl]- 4,5-dihydro-5- (trifluoromethyl)-3- isoxazolyl]-8- isoquinoline- carboxamide |
| 82 | 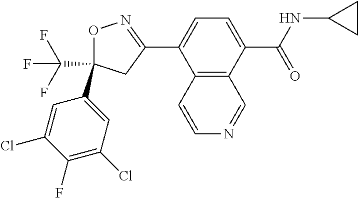 [SMILES: O=C(NC1CC1)c1ccc(C2=NO[C@@](c3cc(Cl)c(F)c(Cl)c3)(C(F)(F)F)C2)c2ccncc12] | S-N- cyclopropyl-5-[5-(3,5- dichloro-4- fluorophenyl)-4,5- dihydro-5- (trifluoromethyl)-3- isoxazolyl]-8- isoquinoline- carboxamide (Compound 82) |
| 99 | 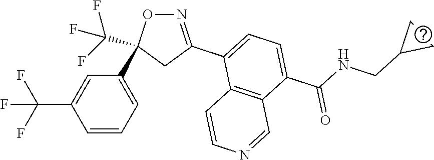 [SMILES: O=C(NCC1CC1)c1ccc(C2=NO[C@@](c3cccc(C(F)(F)F)c3)(C(F)(F)F)C2)c2ccncc12] | S-N- (cyclopropylmethyl)-5- [4,5-dihydro-5- (trifluoromethyl)-5-[3- (trifluoromethyl)phenyl]- 3-isoxazolyl]-8- isoquinoline- carboxamide |

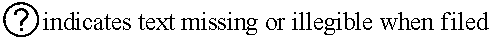

In one embodiment, this disclosure also provides compounds and composition for controlling an invertebrate pest comprising at least one compound of Formula 1. In some embodiments, compounds or compositions disclosed herein further comprise at least one additional component selected from surfactants, solid diluents and liquid diluents. In some embodiments, the compositions disclosed herein optionally further comprise at least one additional biologically active compound or agent.

In one embodiment, this disclosure also provides compounds and compositions for controlling an invertebrate pest comprising a compound of Formula 1, and at least one additional biologically active compound or pest control agent.

In one embodiment, this disclosure provides a method for controlling an invertebrate pest comprising contacting the invertebrate pest or its environment with a biologically effective amount of a compound of Formula 1. This disclosure also relates to such method wherein the invertebrate pest or its environment is contacted with a composition comprising a biologically effective amount of a compound of Formula 1, and at least one additional component selected from the group consisting of surfactants, solid diluents and liquid diluents, said composition optionally further comprising a biologically effective amount of at least one additional biologically active compound or agent.

In one embodiment, this disclosure also relates to such method wherein the invertebrate pest or its environment is contacted with a composition comprising a biologically effective amount of a compound of Formula 1, and at least one additional biologically active compound or pest control agent.

In one embodiment, this disclosure also relates to such method wherein the invertebrate pest or its environment is contacted with a composition comprising a biologically effective amount of a compound of Formula 1 at least one additional biologically active compound or pest control agent and at least one additional component selected from the group consisting of surfactants, solid diluents and liquid diluents. In some embodiments, the environment is soil or plant foliage.

In one embodiment, this disclosure also provides a method for protecting a seed from an invertebrate pest comprising contacting the seed with a biologically effective amount of a compound of Formula 1.

In one embodiment, this disclosure also provides a method for protecting a seed from an invertebrate pest comprising contacting the seed with a biologically effective amount of a compound of Formula 1, and at least one additional biologically active compound or pest control agent.

In one embodiment, this disclosure also provides a method for protecting a seed from an invertebrate pest comprising contacting the seed with a biologically effective amount of a compound of Formula 1, at least one additional biologically active compound or pest control agent, and at least one additional component selected from the group consisting of surfactants, solid diluents and liquid diluents.

In one embodiment, this disclosure provides a soil drench formulation comprising the compounds or compositions disclosed herein.

In one embodiment, the compositions as disclosed herein further comprise liquid fertilizer. In some embodiments, the liquid fertilizer is aqueous-based.

In one embodiment, this disclosure provides a spray composition comprising the compounds or compositions disclosed herein. In some embodiments the spray composition further comprises a propellant.

In one embodiment, this disclosure provides a bait composition comprising the compounds or composition disclosed herein. In one embodiment, the bait composition further comprises one or more food materials.

In one embodiment, the bait composition further comprises an attractant. In one embodiment, the bait composition further comprises a humectant.

In one embodiment, the compounds or compositions disclosed herein are solid compositions, such as dusts, powders, granules, pellets, prills, pastilles, tablets, or filled films. In some embodiments, the compositions disclosed herein are solid compositions and are water-dispersible or water-soluble.

In one embodiment, a liquid or dry formulation comprising the compounds or compositions as disclosed herein for use in a drip irrigation system, furrow during planting, handheld sprayer, backpack sprayer, boom sprayer, ground sprayer, aerial application, unmanned aerial vehicle, or a seed treatment.

In one embodiment, the compounds or compositions as disclosed herein for use in a drip irrigation system, furrow during planting, handheld sprayer, backpack sprayer, boom sprayer, ground sprayer, aerial application, unmanned aerial vehicle, or a seed treatment wherein said formulation is sprayed at an ultra-low volume.

In one embodiment, this disclosure also relates to the treated seed.

Of note is that compounds of this disclosure are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the disclosure. Compounds of this disclosure because of their favorable translocation properties or systemicity in plants also protect foliar or other plant parts which are not directly contacted with a compound of Formula 1 or a composition comprising the compound.

Bioaccumulation of pesticides in non-target organisms is an important safety consideration and it is often desirable to limit the systemic exposure and/or accumulation of pesticides and/or their metabolites in non-target organisms. For example, if a compound is to be applied as an insecticide to a crop plant, it is desirable that the compound does not accumulate in the plasma or fat of a vertebrate animal.

Compounds of Formula 1 may show favorable pharmacokinetic properties in vertebrate animals. In particular, compounds of Formula 1 have been found to have rapid clearance from vertebrate animal plasma/blood and a low distribution into vertebrate animal fat, thus reducing the possibility of unwanted bioaccumulation. Of note is the fluorine atom at the 4-position of the phenyl ring attached to the 5-position of the isoxazoline ring.

The pharmacokinetic properties of compounds of Formula 1 can be measured using a wide variety of assay protocols known in the science of pharmacology. In one illustrative method involving a single oral dose, three male and three female rats each receive a single dose of a test substance via oral gavage. Blood is collected via tail vein at 0.25, 0.5, 1, 2, 4, 8, 12 and 24 h, and then every 24 h thereafter until sacrifice. To process the samples to plasma, blood is collected in tubes containing ethylenediaminetetracetic acid (EDTA) and centrifuged at approximately 3000 rpm to separate plasma from red blood cells. Alternatively, blood is collected using microcapillary tubes and dispensed into tubes containing HPLC water (1:1, v/v). Fat is also collected, homogenized and extracted to determine the concentration of the compound of Formula 1 at sacrifice. The plasma or blood and fat are analyzed for the compound of Formula 1 and/or metabolites, for example, by high-performance liquid chromatography (HPLC) with tandem mass spectrometry detection (LC/MS/MS) to determine the concentration of the test substance. The plasma or blood pharmacokinetic data is analyzed using nonlinear modeling software (e.g., Phoenix® WinNonlin®, Pharsight-A Certara™ Company, St. Louis, Mo., U.S.A.) to determine the plasma/blood half-life of the compound of Formula 1, the time after administration when the maximum plasma/blood concentration is reached (Tₘₐₓ), the maximum plasma/blood concentration (Cₘₐₓ), and the area under the plasma/blood concentration curve (AUC). As analysis of fat requires rat sacrifice, fat data is obtained at single time points (i.e. the time of rat sacrifice). The fat:plasma or fat:blood ratio of the compound of Formula 1 is then determined.

Also noteworthy as embodiments of the present disclosure are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present disclosure are compositions for controlling an invertebrate pest comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the disclosure further include methods for controlling an invertebrate pest comprising contacting the invertebrate pest or its environment with a biologically effective amount of a compound of any of the preceding Embodiments (e.g., as a composition described herein).

Embodiments of the disclosure also include a composition comprising a compound of any of the preceding Embodiments, in the form of a soil drench liquid formulation. Embodiments of the disclosure further include methods for controlling an invertebrate pest comprising contacting the soil with a liquid composition as a soil drench comprising a biologically effective amount of a compound of any of the preceding Embodiments.

Embodiments of the disclosure also include a spray composition for controlling an invertebrate pest comprising a biologically effective amount of a compound of any of the preceding Embodiments and a propellant. Embodiments of the disclosure further include a bait composition for controlling an invertebrate pest comprising a biologically effective amount of a compound of any of the preceding Embodiments, one or more food materials, optionally an attractant, and optionally a humectant. Embodiments of the disclosure also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the disclosure also include methods for protecting a seed from an invertebrate pest comprising contacting the seed with a biologically effective amount of a compound of any of the preceding Embodiments.

Embodiments of the disclosure also include methods for controlling an invertebrate pest comprising contacting the invertebrate pest or its environment with a biologically effective amount of a compound of Formula 1 (e.g., as a composition described herein), provided that the methods are not methods of medical treatment of a human body by therapy.

This disclosure also relates to such methods wherein the invertebrate pest or its environment is contacted with a composition comprising a biologically effective amount of a compound of Formula 1, and at least one additional component selected from the group consisting of surfactants, solid diluents and liquid diluents, said composition optionally further comprising a biologically effective amount of at least one additional biologically active compound or agent, provided that the methods are not methods of medical treatment of a human body by therapy.

The compounds of Formula 1 can be prepared by one or more of the following methods and variations as described in Schemes 1-11. The definitions of substituents in the compounds of Formulae 1-20 below are as defined above in the Summary unless otherwise noted. The following abbreviations may be used: DMF is N,N-dimethylformamide, and DBU is 1,8-diazabicyclo[5.4.0]undec-7-ene.

Compounds of Formula 1 can be prepared from compounds of Formulae 2 or 3 by the general method shown in Scheme 1. In one variation of this method, the compound of Formula 1 is prepared by the coupling of a carboxylic acid chloride of Formula 2 with an appropriate amine compound of Formula of 4. The acid chloride of Formula 2 can be prepared by known methods from the carboxylic acid of Formula 3.

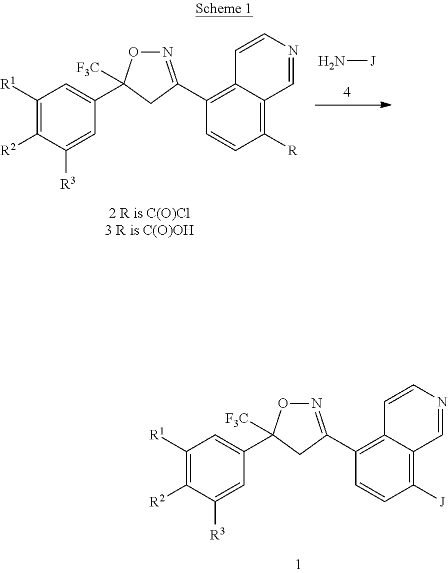

Alternatively, the compound of Formula 1 can be prepared directly from the carboxylic acid of Formula 3 by coupling with an appropriate amine of Formula 4. In this method, the coupling is generally done in the presence of a dehydrating coupling reagent. Coupling reagents useful in this method include dicyclohexyl carbodiimide, 1-(3-dimethylaminopropyl)-3-ethylcarbodiimide and carbonyl diimidazole. Further coupling reagents useful in this method include 1-propanephosphonic acid cyclic anhydride, 1-[bis(dimethylamino)methylene]-1H-1,2,3-triazolo[4,5-b]pyridinium 3-oxide hexafluorophosphate and N-[(dimethylamino)-1H-1,2,3-triazolo-[4,5-b]pyridin-1-ylmethylene]-N-methylmethanaminium hexafluorophosphate N-oxide; these coupling reagents are generally used in the presence of a base such as triethylamine, pyridine, 4-(dimethylamino)pyridine or N,N-diisopropylethylamine. Typical reaction conditions include an anhydrous aprotic solvent such as dichloromethane, tetrahydrofuran or DMF, and a reaction temperature between room temperature and 70° C. The method of Scheme 1 is illustrated in Steps H of Synthesis Example 4.

Compounds of Formula 1 can also be prepared by the method shown in Scheme 2. In this method, an aryl bromide or iodide of Formula 5 is carbonylated and coupled with an appropriate amine compound of Formula of 4.

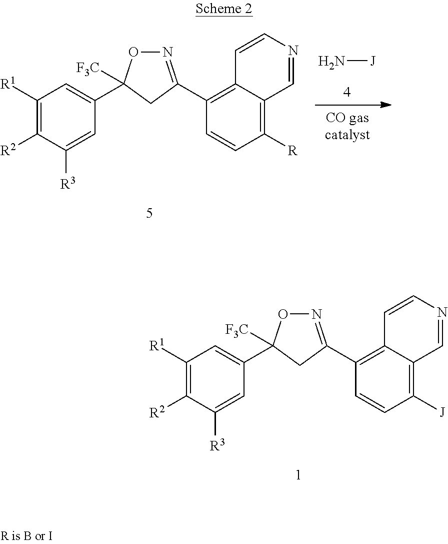

This aminocarbonylation method typically involves treatment of an aryl bromide of Formula 5 (wherein X is Br or I) with an appropriate amine of Formula 4 in the presence of a palladium catalyst under a CO (carbon monoxide) atmosphere. Palladium catalysts useful in this method typically comprise palladium in a formal oxidation state of either 0 (i.e. Pd(0)) or 2 (i.e. Pd(II)). Examples of palladium-containing compounds and complexes useful as catalysts in this method include PdCl₂(PPh₃)₂ (bis(triphenylphosphine)palladium (II) dichloride), Pd(PPh₃)₄ (tetrakis(triphenylphosphine)palladium(0)), Pd(C₅H₇O₂)₂ (palladium(II) acetylacetonate), Pd₂(dba)₃ (tris(dibenzylideneacetone)dipalladium(0)), and [1,1′-bis(diphenylphosphino)ferrocene]dichloropalladium(II). The method of Scheme 2 is generally conducted in a liquid phase, with the palladium catalyst having good solubility in the liquid phase. Useful liquid phase solvents include ethers such as 1,2-dimethoxyethane, amides such as N,N-dimethylacetamide, and non-halogenated aromatic hydrocarbons such as toluene.

The method of Scheme 2 can be conducted over a wide range of temperatures, ranging from about 25 to about 150° C. Of note are temperatures from about 60 and about 110° C., which typically provide faster reaction rates and higher product yields. Literature examples of aminocarbonylation methods include H. Horino et al., Synthesis 1989, 715; and J. J. Li, G. W. Gribble, editors, Palladium in Heterocyclic Chemistry: A Guide for the Synthetic Chemist, 2000. The method of Scheme 2 is illustrated in Step C of Synthesis Example 1.

Compounds of Formula 1 can also be prepared by the method shown in Scheme 3. In this method, a trifluoromethyl ketone of Formula 6 is condensed with a compound of Formula 7, followed by reaction with hydroxylamine to form the isoxazoline ring of the compound of Formula 1.

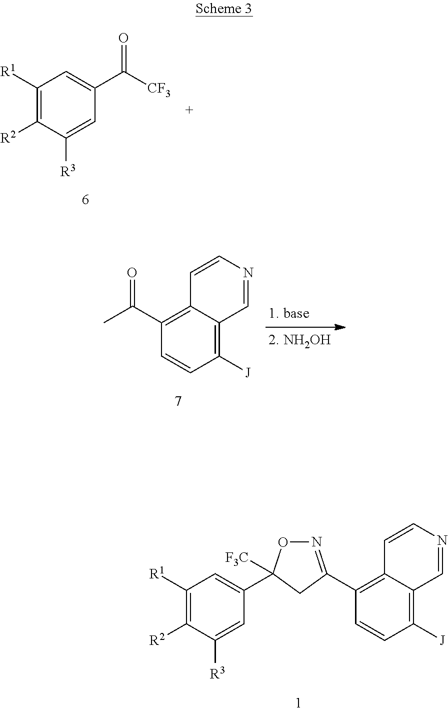

This method involves reaction of the compounds of Formulae 6 and 7 in the presence of a base such as Ca(OH)₂, K₂CO₃ or CsCO₃ in a solvent such as toluene, DMF, MTBE, trifluormethylbenzene, dichloroethane or acetonitrile, or in a mixture of such solvents. The condensation product is subsequently treated with hydroxylamine or a hydroxylamine salt in the presence of base, such as NaOH or LiOH to form the isooxazoline compound of Formula 1. For an example of this method, see G. Annis, WO 2009/126668. The method of Scheme 3 is illustrated in Steps D and E of Synthesis Example 2 and Steps B and C of Synthesis Example 3.

Compounds of Formula 5 can be prepared by the 1,3-dipolar cycloaddition of styrenes of Formula 8 with nitrile oxides derived from oximes of Formula 9 as shown in the method of Scheme 4

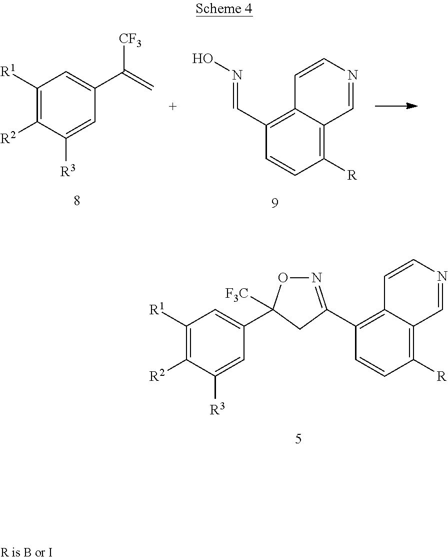

This method typically involves chlorination of the oxime of Formula 9 and subsequent dehydrochlorination to yield an in situ generated nitrile oxide, which then undergoes 1,3-dipolar cycloaddition with the styrene of Formula 8 to afford the compound of Formula 5. In a typical procedure, a chlorinating reagent such as sodium hypochlorite, N-chlorosuccinimide, or chloramine-T is combined with the oxime of Formula 9 in the presence of the styrene of Formula 8. Depending on the reaction conditions, an amine base such as pyridine or triethylamine may be necessary to facilitate the dehydrochlorination reaction. Solvents useful in this method include tetrahydrofuran, diethyl ether, methylene chloride, dioxane, and toluene. Reaction temperatures range from room temperature to the reflux temperature of the solvent. For general procedures for the cycloaddition of nitrile oxides and olefins, see Lee, Synthesis, 1982, 6, 508-509; Kanemasa et al., Tetrahedron, 2000, 56, 1057-1064; EP 1,538,138 A1, as well as references cited within. The preparation of the compound of Formula 9 (wherein R is Br) is known in the art; see Ming Xu et al., Bioorg. Med. Chem. Lett. 2014, 24, 4026.

Compounds of Formula 5 can also be prepared by the method shown in Scheme 5. In this method, a trifluoromethyl ketone of Formula 6 is condensed with a compound of Formula 10, followed by reaction with hydroxylamine to form the isoxazoline ring of the compound of Formula 5.

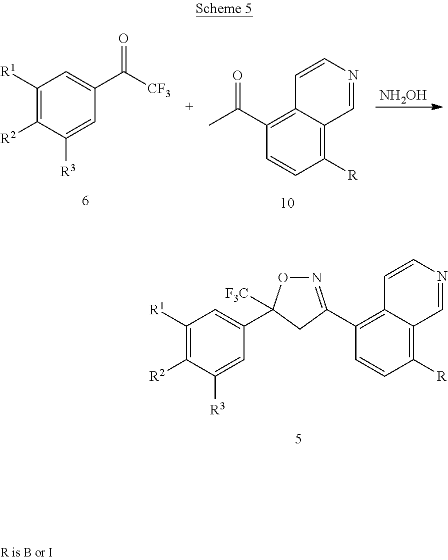

This method is analogous to the method described in Scheme 3. The compound of Formula 10 wherein R is Br is commercially available.

Carboxylic acids of Formula 3 can also be prepared by a method analogous to the method described in Scheme 3. Condensation of the compound of Formula 6 with the compound of Formula 11, followed by cyclization with hydroxyl amine provides the corresponding ester as shown in Scheme 6. Subsequent hydrolysis of the corresponding ester can be accomplished by various procedures known in the art. For example, treatment of the ester with aqueous lithium hydroxide in tetrahydrofuran, followed by acidification, yields the corresponding carboxylic acid of Formula 3. The method of Scheme 6 is illustrated in Steps E, F and G of Synthesis Example 4.

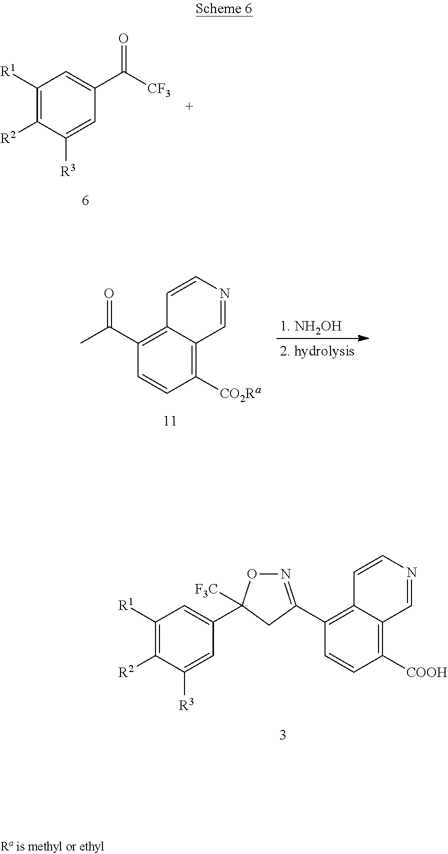

Carboxylic acids of Formula 3 can also be prepared by acidic hydrolysis of amides of Formula 12 as shown in Scheme 7.

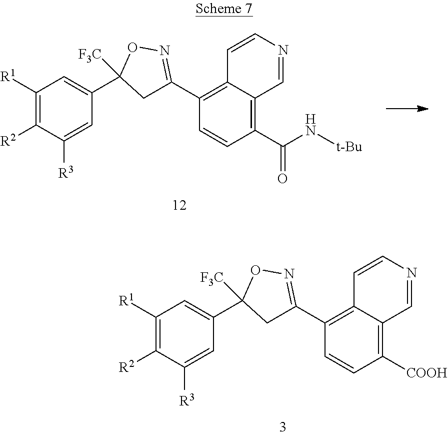

In this method, an amide of Formula 12 is converted to the corresponding carboxylic acid of Formula 3 by procedures known in the art; see, for example, Hoang V. Le, et al. Tetrahedron Lett. 2011, 52(17), 2209. Acids useful for this method include CF₃COOH, H₂SO₄, HOAc, HCl and HBr. Reaction temperatures range from room temperature to the reflux temperature of the solvent. This method is illustrated in Step A of Synthesis Example 3.

Compounds of Formula 11 can be prepared by the method shown in Scheme 8. In this method, an aryl bromide or iodide of Formula 13 is treated with carbon monoxide gas in the presence of a palladium catalyst and coupled with methanol or ethanol to form the compound of Formula of 11 (wherein Ra is methyl or ethyl). This method is similar to the method described in Scheme 2.

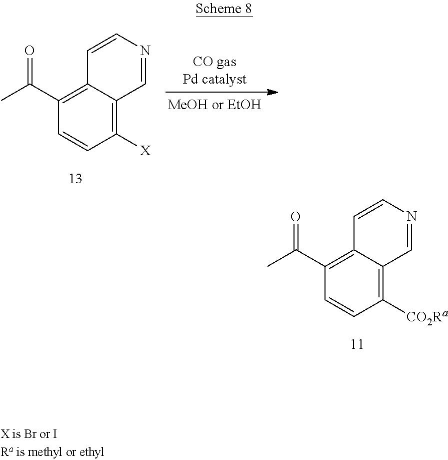

Compounds of Formula 12 can be prepared by the method shown in Scheme 9. This method is similar to the method described in Scheme 3.

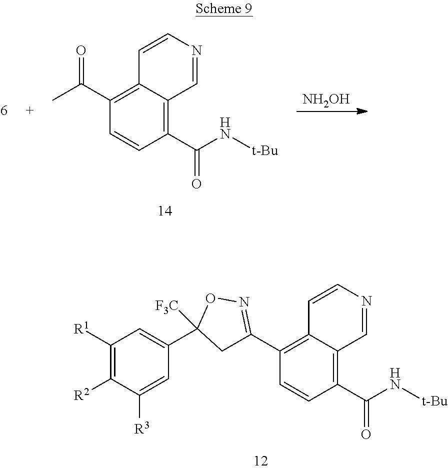

In this method, a trifluoromethyl ketone of Formula 6 is coupled with an amide of Formula 14 and cyclized to provide the compound of Formula 12.

Compounds of Formula 14 can be prepared by the two-step method shown in Scheme 10. In the first step of this method, an amine compound of Formula 15 is diazotized and reacted with a t-butylisocyanide to form a compound of Formula 16.

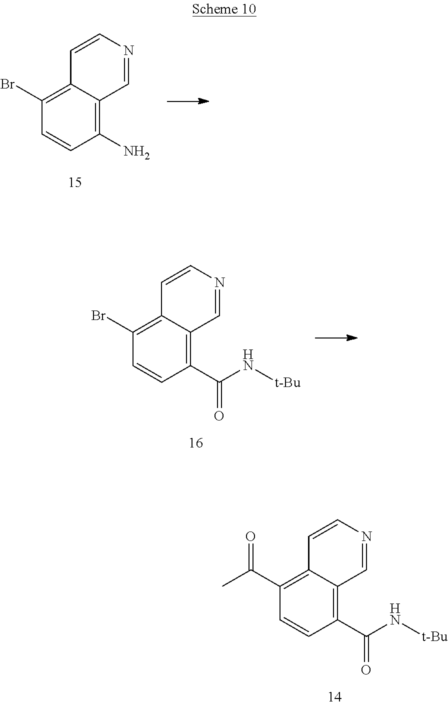 [SMILES: Nc1ccc(Br)c2ccncc12 ; CC(C)(C)NC(=O)c1ccc(Br)c2ccncc12 ; CC(=O)c1ccc(C(=O)NC(C)(C)C)c2cnccc12]

This method of converting an aniline to a diazonium salt and then quenching with an isocyanide to form an amide is known in the literature; see Zhonghua Xia and Qiang Zhu, Org. Lett. 2013, 15(16), 4110, and U. Basavanag, et al. Angew. Chem. Int. Ed. 2013, 52, 7194. The compound of Formula 15 is commercially available. This first step of the method of Scheme 10 is illustrated in Step A of Synthesis Example 2.

The second step of the method of Scheme 10, in which an aryl bromide is converted to a methyl ketone, is well known in the art; see, for example, Youssef, Ei-Ahmad, et al. WO2013/190123; Dan Xu, et al. Tetrahedron Lett. 2008, 49(42), 6104; and Wen Pei, et al. J. Organometallic Chem. 2005, 690(15), 3546. In this step, a compound of Formula 16 is reacted with a vinyl ether such as n-butyl vinyl ether, tributyl(1-ethoxyvinyl)tin or ethyl vinyl ether, in the presence of a palladium catalyst, such as Pd(OAc)₂/Ph₃P(CH₂)₃PPh₃, PdCl₂(PPh₃)₂ or Pd(PPh₃)₄ to provide the coupled product, which is then hydrolyzed with acid to give a compound of Formula 14. The method of Scheme 10 is illustrated in Steps B and C of Synthesis Example 2.

An alternate method for the preparation of the compound of Formula 11 is shown in Scheme 11. In the first step of this method, 2-bromobenzaldehyde 17 is condensed with 2-amino-1,1-dimethoxyethane followed by cyclization with sulfuric acid to yield the bromoisoquinoline of Formula 18. For an example of this Pomerantz-Fritsch reaction, see N. Briet, et al., Tetrahedron, 2002, 58(29), 5761-5766. This procedure may be adapted to prepare other isoquinolines.

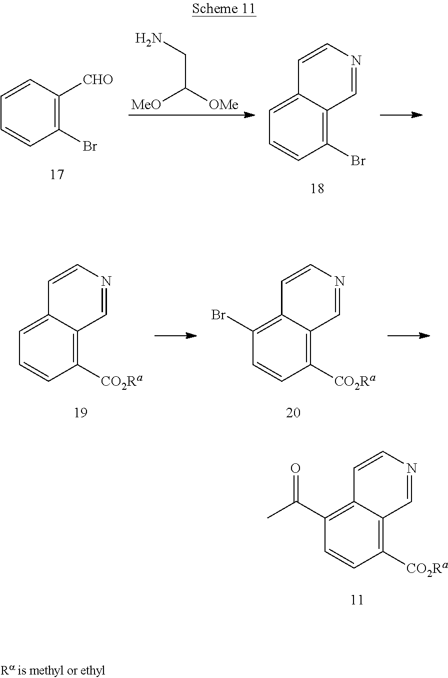

In the second step of this method, the isoquinoline ester of Formula 19 is prepared by treatment of the compound of Formula 18 with carbon monoxide gas in the presence of a palladium catalyst and methanol or ethanol as solvent; see, for example, J. Papillon, et al., J. Med. Chem., 2015, 58(23), 9382-9384. In the third step of this method, the isoquinoline ester of Formula 19 is selectively brominated at the 5-position with N-bromosuccinimide in sulfuric acid/water or with molecular bromine and aluminum chloride to provide the compound of Formula 20; see, for example, W. Brown, et al., Synthesis, 2002, 1, 83-86. In the final step of this method, introduction of the acetyl group can be accomplished by a variety of methods, including the cross coupling of the compound of Formula 20 with tributyl(ethoxyethenyl)stannane in the presence of a palladium catalyst followed by hydrolysis of the vinyl ether to afford the compound of Formula 11; see, for example, N. Sato and N. Narita, Synthesis, 2001, 10, 1551-1555. The method of Scheme 11 is illustrated in Steps A, B, C and D of Synthesis Example 4.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Examples of intermediates useful in the preparation of compounds of this disclosure are shown in Tables I-1 through I-5.

TABLE I-1

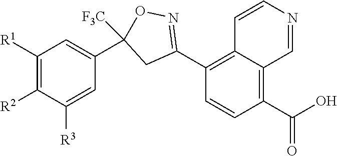

| R¹ | R² | R³ |
|---|---|---|
| Cl | H | Cl |
| Cl | H | CF₃ |
| Cl | F | Cl |
| Cl | F | CF₃ |
| H | F | Cl |
| H | F | CF₃ |
| H | Cl | Cl |
| H | Cl | CF₃ |
| Cl | Cl | Cl |
| Cl | Cl | CF₃ |
| CF₃ | H | CF₃ |
| CF₃ | F | CF₃ |
| CF₃ | Cl | CF₃ |
| H | H | Cl |
| H | H | CF₃ |
| Cl | H | H |
| CF₃ | H | H |
| Cl | F | H |
| CF₃ | F | H |
| Cl | Cl | H |
| CF₃ | Cl | H |

TABLE I-2

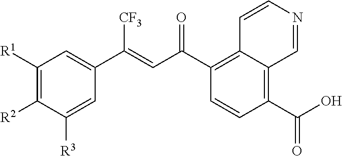

| R¹ | R² | R³ |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| CF₃ | H | CF₃ |
| CF₃ | F | CF₃ |
| CF₃ | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

TABLE I-3

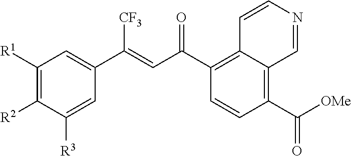

| R¹ | R² | R³ |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| CF₃ | H | CF₃ |
| CF₃ | F | CF₃ |
| CF₃ | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |
| Cl | H | H |

TABLE I-4

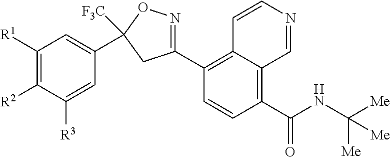

| R¹ | R² | R³ |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| CF₃ | H | CF₃ |
| CF₃ | F | CF₃ |
| CF₃ | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

TABLE I-5

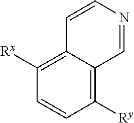

| R^{x} | R^{y} |
|---|---|
| Br | C(O)OH |
| Br | CO₂Me |
| Br | CO₂Et |
| Br | C(O)NH(t-Bu) |
| I | C(O)OH |
| I | CO₂Me |
| I | CO₂Et |
| I | C(O)NH(t-Bu) |
| C(O)Me | Br |
| C(O)Me | I |
| C(O)Me | C(O)OH |
| C(O)Me | CO₂Me |
| C(O)Me | CO₂Et |
| C(O)Me | C(O)NH(t-Bu) |
| CHO | Br |
| CHO | I |
| CHO | C(O)OH |
| CHO | CO₂Me |
| CHO | CO₂Et |
| CHO | C(O)NH(t-Bu) |

Without further elaboration, it is believed that one skilled in the art using the preceding description can utilize the present disclosure to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet. DMF means N,N-dimethylformamide. Compound numbers refer to Index Table A.

### Synthesis Example 1

### Preparation of 5-[5-(3,5-dichloro-4-fluorophenyl)-4,5-dihydro-5-(trifluoromethyl)-3-isoxazolyl]-N-(2-pyrimidinylmethyl)-8-isoquinolinecarboxamide (Compound 4)

### Step A: Preparation of 1-(8-bromo-5-isoquinolinyl)-3-(3,5-dichloro-4-fluorophenyl)-4,4,4-trifluoro-2-buten-1-one

A mixture of 1-(3,5-dichloro-4-fluorophenyl)-2,2,2-trifluoroethanone (1.80 g, 6.39 mmol), 1-(8-bromo-5-isoquinolyl)ethanone (1.00 g, 4.00 mmol, CAS Reg. No. 1890438-87-5)) and cesium carbonate (2.60 g, 8.00 mmol) in toluene (200 mL) was stirred at reflux for 16 hr. The reaction mixture was then cooled and filtered to remove insoluble salts. The filtrate was concentrated and the residue was purified by silica gel column chromatography using hexanes/ethyl acetate as eluent to afford the title compound as a brown oil (0.39 g, 20% yield, 0.79 mmol). ¹H NMR (CDCl₃): 9.69 (s, 1H), 8.74 (d, 1H), 8.43 (d, 1H), 7.89 (d, 1H), 7.85 (d, 1H), 7.38 (s, 1H), 7.19 (s, 1H), 7.17 (s, 1H).

### Step B: Preparation of 8-bromo-5-[5-(3,5-dichloro-4-fluorophenyl)-4,5-dihydro-5-(trifluoromethyl)-3-isoxazolyl]isoquinoline

To a stirred solution of 1-(8-bromo-5-isoquinolinyl)-3-(3,5-dichloro-4-fluorophenyl)-4,4,4-trifluoro-2-buten-1-one (350 mg, 0.71 mmol) and n-tetrabutylammonium bromide (46 mg, 0.14 mmol) was added a solution of sodium hydroxide (284 mg, 71 mmol) and hydroxylamine (0.09 mL, 50% aqueous solution, 1.42 mmol) at 0° C. After stirring at 0° C. for 1 hr, the reaction mixture was partitioned between water and ethyl acetate, the layers were separated, and the aqueous layer was washed again with ethyl acetate. The combined organic layers were washed with water and brine, dried (Na₂SO₄), and concentrated under reduced pressure. The residue was purified by silica gel column chromatography using hexanes/ethyl acetate as eluent to afford the title compound as a yellow solid (290 mg, 80% yield, 0.57 mmol). ¹H NMR (CDCl₃): 9.69 (s, 1H), 8.80 (d, 1H), 8.74 (d, 1H), 7.87 (d, 1H), 7.63 (s, 1H), 7.62 (s, 1H), 7.54 (d, 1H), 4.27 (d, 1H), 3.90 (d, 1H).

### Step C: Preparation of 5-[5-(3,5-dichloro-4-fluorophenyl)-4,5-dihydro-5-(trifluoromethyl)-3-isoxazolyl]-N-(2-pyrimidinylmethyl)-8-isoquinolinecarboxamide

A mixture of 8-bromo-5-[5-(3,5-dichloro-4-fluorophenyl)-4,5-dihydro-5-(trifluoromethyl)-3-isoxazolyl]isoquinoline (180 mg, 0.35 mmol), 2-aminomethylpyrimidine hydrochloride (154 mg, 1.41 mmol), [1,1′-bis(diphenylphosphino)ferrocene]-dichloropalladium(II) (29 mg, 0.04 mmol) and triethylamine (0.49 mL, 3.5 mmol) in toluene (10 mL) was stirred at 80° C. under one atmosphere of carbon monoxide for 6 hr. The reaction mixture was then filtered through a short pad of Celite®, rinsed with ethyl acetate, and the filtrate was concentrated. The resulting residue was purified by silica gel column chromatography using ethyl acetate/methanol as eluent to afford the title compound, a compound of this disclosure, as a yellow solid (88 mg, 45% yield, 0.16 mmol). ¹H NMR (DMSO-d₆): 9.83 (s, 1H), 9.46 (t, 1H), 8.86 (d, 2H), 8.71 (s, 2H), 8.17 (d, 1H), 7.92 (d, 1H), 7.90 (s, 1H), 7.88 (s, 1H), 7.47 (t, 1H), 4.78 (d, 2H), 4.62 (d, 1H), 4.58 (d, 1H).

### Synthesis Example 2

### Preparation of 5-[5-(3,5-dichloro-4-fluorophenyl)-4,5-dihydro-5-(trifluoromethyl)-3-isoxazolyl]-N-(1,1-dimethylethyl)-8 soquinolinecarboxamide (compound 2)

### Step A: Preparation of 5-bromo-N-(1,1-dimethylethyl)-8-isoquinolinecarboxamide

To a stirred suspension of 5-bromo-8-isoquinolinamine (8.0 g, 35.86 mmol) and HBF₄ (10.67 mL, 50% in aqueous solution, 58.32 mmol) in water (25 mL) was added slowly a solution of NaNO₂ (2.73 g, 39.56 mmol) in water (15 mL) at 0° C. The reaction mixture was stirred at 0° C. for 30 min, and then stirred at room temperature for 1 hr. The brown insoluble solid was collected via filtration and rinsed with a small amount of water. This solid was transferred to another reaction flask and acetone (90 mL) was added, followed by the slow addition of t-butylisocyanide (12.15 mL, 107.49 mmol), then cesium carbonate (35 g, 107.49 mmol). After stirring at room temperature for 1.5 hr, the reaction mixture was concentrated to remove most of the acetone solvent and excess t-butylisocyanide. The resulting residue was partitioned between water and ethyl acetate. The phases were separated, the organic layer was washed with brine, dried (Na₂SO₄), and concentrated under reduced pressure. The residue was purified by silica gel column chromatography using hexanes/ethyl acetate as eluent to afford the title compound as a brown solid (7.60 g, 69% yield, 24.76 mmol). ¹H NMR (CDCl₃): 9.63 (s, 1H), 8.68 (d, 1H), 8.01 (d, 1H), 7.94 (d, 1H), 7.50 (d, 1H), 5.90 (br s, 1H), 1.55 (s, 9H).

### Step B: Preparation of N-(1,1-dimethylethyl)-5-(1-ethoxyethenyl)-8-isoquinolinecarboxamide

To a stirred mixture of 5-bromo-N-(1,1-dimethylethyl)-8-isoquinolinecarboxamide (2.95 g, 9.60 mmol) in toluene (50 mL) was added (1-ethoxyethenyl)tributylstannane (4.85 mL, 14.39 mmol) and tetrakis(triphenylphosphine)palladium (0) (1.1 g, 0.96 mmol). The reaction mixture was heated to reflux under a nitrogen atmosphere for 4 hr. The reaction mixture was then concentrated, and the residue purified by silica gel column chromatography using hexanes/ethyl acetate as eluent to afford the title compound as a brown solid (2.44 g, 85% yield, 8.17 mmol). ¹H NMR (CDCl₃): 9.64 (s, 1H), 8.58 (d, 1H), 7.99 (d, 1H), 7.704 (d, 1H), 7.60 (d, 1H), 5.80 (br s, 1H), 4.55 (d, 1H), 4.40 (d, 1H), 4.03 (q, 2H), 1.54 (s, 9H), 1.43 (t, 3H).

### Step C: Preparation of 5-acetyl-N-(1,1-dimethylethyl)-8-isoquinolinecarboxamide

To a stirred solution of N-(1,1-dimethylethyl)-5-(1-ethoxyethenyl)-8-isoquinolinecarboxamide (2.44 g, 8.17 mmol) in toluene (50 mL) was added concentrated HCl (10 mL) and water (10 mL). After stirring at room temperature for 1 hr, the reaction mixture was partitioned between water and ethyl acetate, the pH of the aqueous layer was adjusted to 8, and the aqueous layer was separated and further extracted with ethyl acetate. The combined organic extracts were washed with brine, dried (Na₂SO₄), and concentrated under reduced pressure. The residue was purified by silica gel column chromatography using hexanes/ethyl acetate as eluent to afford the title compound as a yellow solid (1.80 g, 80% yield, 6.67 mmol). ¹H NMR (CDCl₃): 9.62 (s, 1H), 8.64 (d, 1H), 8.58 (d, 1H), 8.12 (d, 1H), 7.65 (d, 1H), 5.89 (s, br. 1H), 2.75 (s, 3H), 1.56 (s, 9H).

### Step D: Preparation of 5-[3-(3,5-dichloro-4-fluorophenyl)-4,4,4-trifluoro-1-oxo-2-buten-1-yl]-N-(1,1-dimethylethyl)-8-isoquinolinecarboxamide

To a stirred solution of 5-acetyl-N-(1,1-dimethylethyl)-8-isoquinolinecarboxamide (0.20 g, 0.74 mmol) in 1,2-dichloroethane (5 mL) was added 1-(3,5-dichloro-4-fluoro-phenyl)-2,2,2-trifluoroethanone (0.39 g, 1.48 mmol), K₂CO₃ (0.13 g, 0.96 mmol) and triethylamine (0.14 mL, 0.96 mmol). The reaction mixture was heated to 100° C. and stirred for 16 hrs under a nitrogen atmosphere. The reaction mixture was then cooled and concentrated. The residue was partitioned between water and ethyl acetate, the layers were separated, and the aqueous layer was washed again with ethyl acetate. The combined organic layers were washed with brine, dried (Na₂SO₄), and concentrated under reduced pressure. The residue was purified by silica gel column chromatography using hexanes/ethyl acetate as eluent to afford the title compound as a brown oil (0.22 g, 58% yield, 0.43 mmol). ¹H NMR (CDCl₃): 9.51 (s, 1H), 8.58 (d, 1H), 8.28 (d, 1H), 7.93 (d, 1H), 7.56 (d, 1H), 7.38 (s, 1H), 7.16 (s, 1H), 7.15 (s, 1H), 6.07 (br s, 1H), 2.75 (s, 3H), 1.54 (s, 9H).

### Step E: Preparation of 5-[5-(3,5-dichloro-4-fluorophenyl)-4,5-dihydro-5-(trifluoromethyl)-3-isoxazolyl]-N-(1,1-dimethylethyl)-8-isoquinolinecarboxamide

To a stirred solution of 5-[3-(3,5-dichloro-4-fluorophenyl)-4,4,4-trifluoro-1-oxo-2-buten-1-yl]-N-(1,1-dimethylethyl)-8-isoquinolinecarboxamide (0.22 g, 0.43 mmol) in 1,2-dimethoxyethethane (5 mL) and water (1 mL) was added hydroxylamine hydrochloride (30 mg, 0.86 mmol) and lithium hydroxide monohydrate (72 mg, 1.72 mmol). The reaction mixture was stirred at room temperature for 1 hr, and then partitioned between water and ethyl acetate. The layers were separated, and the aqueous layer was washed again with ethyl acetate. The combined organic layers were washed with brine, dried (Na₂SO₄), and concentrated under reduced pressure. The residue was purified by silica gel column chromatography using hexanes/ethyl acetate as eluent to afford the title compound, a compound of this disclosure, as a brown oil (0.22 g, 58% yield, 0.43 mmol). ¹H NMR (CDCl₃): 9.58 (s, 1H), 8.71 (d, 1H), 8.63 (d, 1H), 7.64 (m, 4H), 6.02 (br s, 1H), 4.25 (d, 1H), 3.90 (d, 1H), 1.55 (s, 9H).

### Synthesis Example 3

### Preparation of 5-[5-(3,5-dichloro-4-fluorophenyl)-4,5-dihydro-5-(trifluoromethyl)-3-isoxazolyl]-N-[1-(methylsulfonyl)-3-azetidinyl]-8-isoquinolinecarboxamide (compound 21)

### Step A: Preparation of 5-acetyl-N-[1-(methylsulfonyl)-3-azetidinyl]-8-isoquinolinecarboxamide

A mixture of 5-acetyl-N-(1,1-dimethylethyl)-8-isoquinolinecarboxamide (0.20 g, 0.74 mmol) in acetic acid (2 mL) and hydrobromic acid (2 mL, 48% aqueous solution) was stirred at 120° C. for 12 hr. The reaction mixture was then cooled to room temperature and concentered under reduced pressure. The residue was dissolved in DMF (3 mL), and to this solution was added 1-methylsulfonylazetidin-3-amine (213 mg, 1.4 mmol), triethylamine (0.4 mL) and HATU (424 mg, 1.11 mmol). The reaction mixture was stirred at room temperature overnight, and was then adsorbed onto Celite® and purified by reverse phase chromatography with H₂O/CH₃CN/MeOH as eluent to afford the title compound as a white solid (68 mg, 26% yield, 0.20 mmol). ¹H NMR (CDCl₃): 9.55 (s, 1H), 9.51 (d, 1H), 8.66 (d, 1H), 8.49 (d, 1H), 8.47 (d, 1H), 7.89 (d, 1H), 4.81 (m, 1H), 4.21 (dd, 2H), 3.98 (dd, 2H), 3.06 (s, 3H), 2.77 (s, 3H).

### Step B: Preparation of 5-[3-(3,5-dichloro-4-fluorophenyl)-4,4,4-trifluoro-1-oxo-2-buten-1-yl]-N-[1-(methyl sulfonyl)-3-azetidinyl]-8-isoquinolinecarboxamide

The title compound was prepared by a procedure analogous to Step D of Synthesis Example 2 as a yellow oil (40 mg, 47% yield). ¹H NMR (CDCl₃): 9.56 (s, 1H), 8.62 (d, 1H), 8.31 (d, 1H), 7.97 (d, 1H), 7.68 (d, 1H), 7.39 (d, 1H), 7.28 (br s, 1H), 7.18 (s, 1H), 7.17 (s, 1H), 5.00 (m, 1H), 4.26 (dd, 2H), 4.07 (dd, 2H), 2.89 (s, 3H).

### Step C: Preparation of 5-[5-(3,5-dichloro-4-fluorophenyl)-4,5-dihydro-5-(trifluoromethyl)-3-isoxazolyl]-N-[1-(methylsulfonyl)-3-azetidinyl]-8-isoquinolinecarboxamide

The title compound, a compound of this disclosure, was prepared by a procedure analogous to Step E of Synthesis Example 2 as a white solid. ¹H NMR (CDCl₃): 9.54 (s, 1H), 8.67 (d, 1H), 8.58 (d, 1H), 7.84 (m, 5H), 7.36 (d, 1H), 4.97 (m, 1H), 4.26 (d, 1H), 4.24 (dd, 2H), 4.08 (dd, 2H), 3.90 (d, 1H), 2.90 (s, 3H).

### Synthesis Example 4

### Preparation of N-(cyclopropylmethyl)-5-[4,5-dihydro-5-(trifluoromethyl)-5-[3-(trifluoromethyl)phenyl]-3-isoxazolyl]-8-isoquinolinecarboxamide (compound 17)

### Step A: Preparation of 8-bromoisoquinoline

A solution of 2-bromobenzaldehyde (50 g, 270.27 mmol, 1 eq) and 2,2-dimethoxyethylamine (34.4 g, 324.32 mmol, 1.2 eq) in toluene (300 mL) was azeotroped at reflux for 2 hr using a Dean-Stark apparatus. The progress was monitored by TLC. The reaction mixture was concentrated under reduced pressure to obtain a pale brown viscous oil. The crude compound was dissolved in dichloromethane (400 mL) and AlCl₃ (118.6 g, 891.89 mmol, 3.3 eq) was added portionwise at 0° C. The reaction was gradually heated to 45° C. and stirred for 16 hr at the same temperature. The reaction was monitored by TLC. Upon completion, it was poured into cold water (500 mL) and extracted with dichloromethane (2×500 mL). The organic layer was washed with brine solution, dried over anhydrous Na₂SO₄, and concentrated under reduced pressure to afford a purple-colored solid. The crude compound was purified by silica gel (60-120) column chromatography and eluted with 50% ethyl acetate in petroleum ether to give a pale yellow solid 8-bromoisoquinoline (40 g, 71.5% yield). ¹H NMR (400 MHz CDCl₃), 9.621 (s, 1H), 8.623-8.609 (d, 1H, J=5.6), 7.860-7.842 (d, 1H, J=7.2), 7.803-7.782 (d, 1H. J=8.4), 7.633-7.619 (d, 1H, J=5.6), 7.552-7.513, (t, 1H, J15.6).

### Step B: Preparation of methyl isoquinoline-8-carboxylate

A solution of 8-bromoisoquinoline (13 g, 63.106 mmol, 1 eq, from Step A) in MeOH (130 mL) was charged in a steel bomb and Et₃N (19.15 g, 189.32 mmol, 3.0 eq) was added. The solution was purged with nitrogen for 10-15 min and PdCl₂(dppf) (4.61 g, 6.310 mmol, 0.1 eq) was added. After which, the reaction mixture was passed with CO gas (150 psi), heated to 100° C. for 16 hr. After TLC indication, the reaction mixture was cooled to room temperature, passed through a pad of celite, and washed with ethyl acetate (500 mL). The combined organic layers were washed with water (200 mL) then a brine solution, dried over anhydrous Na₂SO₄, and concentrated under reduced pressure. The resulting crude solid was purified by silica gel (60-120) column chromatography, eluted with 30% pet ether/ethyl acetate to give a pale yellow solid methyl isoquinoline-8-carboxylate (10.2 g, 86.80%), ¹H NMR (400 MHz, CDCl₃); 10.23 (s, 1H), 8.629-8.615 (d, 1H, J=5.6), 8.286-8.268 (d, 1H, J=7.2), 8.011-7.991 (d, 1H, J=8.0), 7.739-7.720 (d, 1H, J=7.6), 7.700-7.686, (d, 1H, J=5.6), 4.100 (s, 3H).

### Step C: Preparation of methyl 5-bromo-isoquinoline-8-carboxylate

To a solution of methyl isoquinoline-8-carboxylate (22 g, 118.279 mmol, 1 eq, from Step B) in H₂SO₄ (200 mL) was added N-bromosuccinimide (27.36 g, 153.76 mmol, 1.3 eq.) portionwise at 0° C. The reaction mixture was warmed to room temperature, stirred for 16 hr, and monitored by TLC. The reaction mixture was poured into ice cold water (2 L) and basified (pH 8) with NH₄OH solution. The resulting precipitate was collected (1′ crop). The aqueous layer was extracted with ethyl acetate (1 L), and the organic layer was washed with brine solution, dried over anhydrous Na₂SO₄ and concentrated under reduced pressure (2^{nd} crop). The combined crude crops were triturated with n-pentane to give title compound methyl 5-bromo-isoquinoline-8-carboxylate (22 g, 70.31% yield) as a pale brown solid. ¹H NMR (400 MHz, CDCl₃); 10.281 (s, 1H), 8.736-8.722 (d, 1H, J=5.6), 8.117-8.098 (d, 1H, J=7.6), 8.083-8.068 (d, 1H, J=6.0), 8.033-8.013 (d, 1H, J=8.0), 4.010 (s, 3H).

### Step D: Preparation of methyl 5-acetyl-isoquinoline-8-carboxylate

To a solution of methyl 5-bromo-isoquinoline-8-carboxylate (22 g, 82.706 mmol, 1 eq, from Step C) in toluene (220 mL) was added tributyl(1-ethoxyvinyl)tin (38.8 g, 107.51 mmol, 1.3 eq) and purged with N2 for 15 min. PdCl₂(PPh₃) (5.8 g, 8.270 mmol, 0.1 eq) was added at room temperature, and the resulting mixture was stirred at 100° C. for 16 hr. The reaction was monitored by TLC. Upon completion, the mixture was cooled to room temperature and 1 N HCl (55 mL) was added before stirring at room temperature for an additional 2 hr. The reaction mixture was neutralized (pH 7) with aq. NaHCO₃ solution (300 mL) and extracted with ethyl acetate (2 L). The combined organic layers were washed with brine, dried over anhydrous Na₂SO₄ and concentrated. The crude solid was purified by silica gel (60-120) column chromatography, eluted with 70% ethyl acetate in petroleum ether to provide the title compound (16.5 g, 87%) as a pale-yellow solid. ¹H NMR (400 MHz, CDCl₃); 10.207-10.204 (d, 1H, J=1.2), 8.707-8.691 (d, 1H, J=6.4), 8.509-8.492 (d, 1H, J=6.8), 8.235-8.215 (d, 1H, J=8.0), 8.134-8.115 (d, 1H, J=7.6) 4.079 (s, 3H), 2.779 (s, 3H).

### Step E: Preparation of methyl 4-[(Z)-4,4,4-trifluoro-3-[3-(trifluoromethyl)phenyl]but-2-enoyl]naphthalene-1-carboxylate

To a solution of methyl 5-acetyl-isoquinoline-8-carboxylate (10 g, 43.668 mmol, 1 eq, from Step D) in 2-methyltetrahydrofuran was added molecular sieves (10 g), K₂CO₃ (30 g, 218.340 mmol, 5 eq) and 2,2,2-trifluoro-1-(3-trifluoromethyl)phenyl)ethanone (18 g, 86.60 mmol, 2 eq). The resulting reaction mixture was stirred at 90° C. for 16 hr under a N2 atmosphere. Upon indication by TLC, the reaction was cooled to room temperature, passed through a pad of celite, washed with ethyl acetate (1 L) and concentrated under reduced pressure. The obtained crude oil was purified by silica gel (60-120) column chromatography (5% MeOH/dichloromethane) to provide an off-white solid of the title compound (8 g, 40.48%). ¹H NMR (400 MHz DMSO-D₆); 9.890 (s, 1H), 8.624-8.609 (d, 1H, J=6.0), 8.344-8.325 (d, 1H, J=7.6), 8.142-8.117 (m, 2H, J=10.0), 7.770 (s, 1H,) 7.542 (s, 1H) 7.525 (s, 1H), 7.480 (s, 1H), 7.423-7.385 (t, 1H, J=15.2), 4.001 (s, 3H).

### Step F: Preparation of methyl 5-[5-(trifluoromethyl)-5-[3-(trifluoromethyl)phenyl]-4H-isoxazol-3-yl]isoquinoline-8-carboxylate

To a solution of compound methyl 4-[(Z)-4,4,4-trifluoro-3-[3-(trifluoromethyl)phenyl]but-2-enoyl]naphthalene-1-carboxylate (8 g, 17.660 mmol, 1 eq, from Step E) in THF (100 mL) was added (NH₂OH)₂—H₂SO₄ (1.44 g, 8.830 mmol, 0.5 eq) at 0° C. followed by the addition of 50% aq. NaOH solution (1.5 g, 38.852 mmol, 2.2 eq). The resulting reaction mixture was stirred at room temperature for 3 hr. The reaction mixture was concentrated to yield compounds methyl 5-[5-(trifluoromethyl)-5-[3-(trifluoromethyl)phenyl]-4H-isoxazol-3-yl]isoquinoline-8-carboxylate: 5-[5-(trifluoromethyl)-5-[3-(trifluoromethyl)phenyl]-4H-isoxazol-3-yl]isoquinoline-8-carboxylic acid (1:1 mixture) as pale yellow oil (7 g, 84.64%), which was taken directly to the next step.

### Step G: Preparation of 5-[5-(trifluoromethyl)-5-[3-(trifluoromethyl)phenyl]-4H-isoxazol-3-yl]isoquinoline-8-carboxylic acid

To a solution of compounds methyl 5-[5-(trifluoromethyl)-5-[3-(trifluoromethyl)phenyl]-4H-isoxazol-3-yl]isoquinoline-8-carboxylate: 5-[5-(trifluoromethyl)-5-[3-(trifluoromethyl)phenyl]-4H-isoxazol-3-yl]isoquinoline-8-carboxylic acid (1:1, 7 g, 1 eq., from Step F) in THF (20 mL), H₂O (5 mL) was added LiOH (2.5 g, 59.82 mmol, 4.0 eq.) at 0° C. Then reaction mixture was stirred at room temperature for 3 hr. After monitoring by TLC, the reaction mixture was concentrated under vacuum, acidified with 1 N HCl (pH 5), and the resulting precipitated was collected, washed with water (500 mL) and dried under reduced pressure to yield compound 5-[5-(trifluoromethyl)-5-[3-(trifluoromethyl)phenyl]-4H-isoxazol-3-yl]isoquinoline-8-carboxylic acid as an off-white solid (5 g, 73.63%). ¹H NMR (400 MHz, DMSO-D₆); 10.180 (s, 1H) 8.767-8.725 (m, 2H, J=16.8), 8.295-8.230 (m, 2H, J=26), 8.011-7.991 (d, 1H, J=8.0), 7.942-7.923 (m, 2H) 7.845-7.806 (t, 1H, J=15.6), 4.726-4.680 (d, 1H, J-18.4), 4.591-4.545 (d, 1H, J-18.4).

### Step H: Preparation of N-(cyclopropylmethyl)-5-[4,5-dihydro-5-(trifluoromethyl)-5-[3-(trifluoromethyl)phenyl]-3-isoxazolyl]-8-isoquinolinecarboxamide

To a solution of 5-[5-(trifluoromethyl)-5-[3-(trifluoromethyl)phenyl]-4H-isoxazol-3-yl]isoquinoline-8-carboxylic acid (0.8 g, 1.766 mmol, 1 eq, from Step G) in DMF (5 mL) were added HATU (1.0, 2.624 mmol, 1.5 eq), DIPEA (0.68 g, 5.286 mmol, 3 eq), cyclopropanemethanamine (0.15 g, 2.114 mmol, 1.2 eq) and stirred at RT for 16 hr. The reaction mixture was poured into ice cold water (50 mL). The resulting precipitate was collected, washed with water (20 mL) dried under reduced pressure, and yielded the title product (200 mg, 22.39%) as racemic mixture. ¹H NMR (400 MHz, DMSO-d6), 9.563 (s, 1H), 8.976-8.948 (t, 1H, J=11.2), 8.724-8.686 (m, 2H, J=15.2), 8.190-8.170 (d, 1H, J=8.0), 8.015-7.995 (d, 1H, J=8.0), 7.945-7.923 (d, 2H, J=8.8), 7.847-7.790 (m, 2H, J=22.8), 4.714-4.668 (d, 1H, J=18.4), 4.580-4.535 (d, 1H, J=18.0), 3.322-3.253 (t, 2H, J=27.3), 1.135-1.097 (m, 2H, J=15.2), 0.527-0.482 (m, 2H, J=18.0), 0.319-0.281 (m, 2H, J=15.2).

By the procedures described herein together with methods known in the art, the following compounds of Table 2 can be prepared. The following abbreviations are used in the Tables which follow: Me means methyl.

TABLE 2

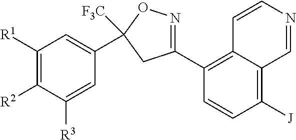

| | | |
|---|---|---|
| R¹ | R² | R³ |

J is J-1 and R⁴ is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-1 and R⁴ is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-1 and R⁴ is cyclopropyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-1 and R⁴ is isopropyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-1 and R⁴ is —CH₂(cyclopropyl)

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-1 and R⁴ is —CH₂CH(CH₃)₂

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is H, and R⁶ is OMe

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is H, and R⁶ is SMe

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is H, and R⁶ is S(O)Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is H, and R⁶ is SO₂Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is Me, and R⁶ is OMe

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is Me, and R⁶ is SMe

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is Me, and R⁶ is S(O)Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-2, R⁵ is Me, and R⁶ is SO₂Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-3, R⁷ is H, and Z is 2-pyridinyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-3, R⁷ is H, and Z is 2-pyrimidinyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-3, R⁷ is Me, and Z is 2-pyridinyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-3, R⁷ is Me, and Z is 2-pyrimidinyl

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is H, and R⁹ is H

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is H, and R⁹ is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is H, and R⁹ is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is H, and R⁹ is n-Pr

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is H, and R⁹ is i-Pr

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is Me, and R⁹ is H

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is Me, and R⁹ is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is Me, and R⁹ is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-4, R⁸ is Me, and R⁹ is n-Pr

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is H, R¹¹ is H, and R¹² is H

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is H, R¹¹ is H, and R¹² is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is H, R¹¹ is H, and R¹² is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is H, R¹¹ is H, and R¹² is CH₂CF₃

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R^{10 is} H, R¹¹ is H, and R¹² is CH₂CN

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is Me, R¹¹ is H, and R¹² is H

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is Me, R¹¹ is H, and R¹² is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is Me, R¹¹ is H, and R¹² is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is Me, R¹¹ is H, and R¹² is CH₂CF₃

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-5, R¹⁰ is Me, R¹¹ is H, and R¹² is CH₂CN

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-6 and R¹³ is H

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-6 and R¹³ is Me

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-6 and R¹³ is Et

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

J is J-6 and R¹³ is CH₂CF₃

| | | |
|---|---|---|
| Cl | H | Cl |
| Cl | H | CF₃ |
| Cl | F | Cl |
| Cl | F | CF₃ |
| Cl | Cl | Cl |
| Cl | Cl | CF₃ |
| H | H | Cl |
| H | H | CF₃ |
| H | F | Cl |
| H | F | CF₃ |
| H | Cl | Cl |
| H | Cl | CF₃ |
| Cl | H | H |
| CF₃ | H | H |
| Cl | F | H |
| CF₃ | F | H |
| Cl | Cl | H |
| CF₃ | Cl | H |

Specific compounds of Formula 1, prepared by the methods and variations as described in preceding Schemes 1-11 and Synthesis Examples 1-4, are shown in the Index Tables below. The following abbreviations are used in Index Table A: i-Pr means iso-propyl, n-Pr means n-propyl, c-Pr means cyclo-propyl, i-Bu means iso-butyl, c-Bu means cyclo-butyl, s-Bu means sec-butyl, t-Bu means tert-butyl, Me means methyl, Et means ethyl and Ph means phenyl. The abbreviation “Cmpd.” stands for “Compound”, and the abbreviation “Ex.” stands for “Example” and is followed by a number indicating in which example the compound is prepared. For mass spectral data (AP⁺ (M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

INDEX TABLE A

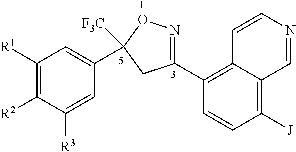

| Cmpd. No. | R¹ | R² | R³ | J | MS data | m.p. (° C.) |
|---|---|---|---|---|---|---|
| 1 | Cl | H | Cl | 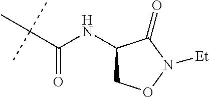 | 567 | |
| 2 | Cl | F | Cl | —C(O)NH(t-Bu) | 528 | |
| 3 | Cl | F | Cl | —C(O)NHCH₂(cyclopropyl) | 526 | |
| 4 | Cl | F | Cl | 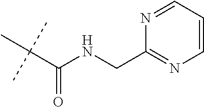 | 564 | |
| 5 | Cl | F | Cl | 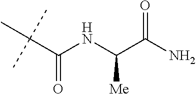 | 543 | |
| 6 | Cl | F | Cl | 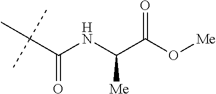 | 558 | |
| 7 | Cl | F | Cl | 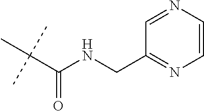 | 564 | |
| 8 | Cl | F | Cl | 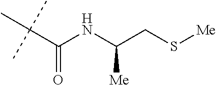 | 560 | |
| 9 | Cl | F | Cl | 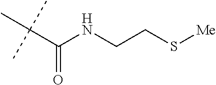 | 546 | |
| 10 | Cl | F | Cl | 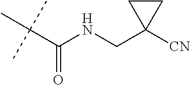 | 551 | |
| 11 | Cl | F | Cl | 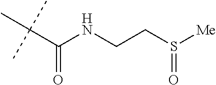 | 562 | |
| 12 | Cl | F | Cl | 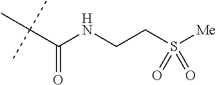 | 578 | |
| 13 | Cl | F | Cl | 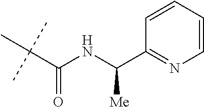 | 577 | |
| 14 | Cl | F | Cl | 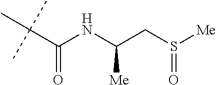 | 576 | |
| 15 | Cl | F | Cl | —C(O)NHCH₂CN | 511 | |
| 16 | H | H | CF₃ | —C(O)NH(cyclopropyl) | 494 | |
| 17 | H | H | CF₃ | —C(O)NHCH₂(cyclopropyl) | 508 | |
| 18 | H | H | CF₃ | 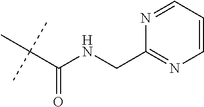 | 546 | |
| 19 | H | H | CF₃ | 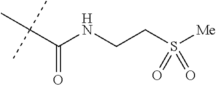 | 560 | |
| 20 | Cl | F | Cl | 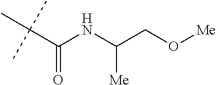 | 544 | |
| 21 | Cl | F | Cl | 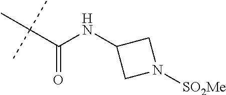 | 605 | |
| 22 | H | H | CF₃ | 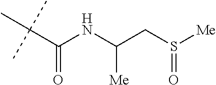 | 558 | |
| 23 | Cl | F | Cl | —C(O)NH(cyclopropyl) | 512 | |
| 24 | H | H | CF₃ | —C(O)NHCH₂C(O)NH₂ | 511 | |
| 25 | H | H | CF₃ | 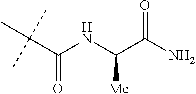 | 525 | |
| 26 | Cl | F | Cl | 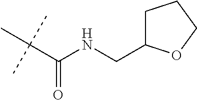 | 556 | |
| 27 | Cl | F | Cl | —C(O)NHMe | 486 | |
| 28 | Cl | F | Cl | —C(O)NHCH(CH₃)₂ | 514 | |
| 29 | H | H | CF₃ | 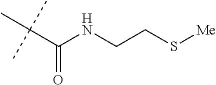 | 528 | |
| 30 | H | H | CF₃ | 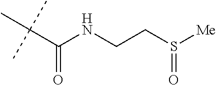 | 544 | |
| 31 | CF₃ | H | H | —C(═O)NH(CH₂)₂S(═O)CH₂CF₃ | 612 | |
| 32 | Cl | F | Cl | —C(═O)NH-3-furanyl | 542 | |
| 33 | Cl | F | Cl | —C(═O)NH-s-Bu | 528 | |
| 34 | Cl | H | CF₃ | —C(═O)NH(CH₂)₂SCH₃ | 562 | |
| 35 | Cl | H | CF₃ | —C(═O)NH-c-Pr | 528 | |
| 36 | Cl | H | CF₃ | —C(═O)NHCH₂-c-Pr | 542 | |
| 37 | Cl | H | CF₃ | —C(═O)NHCH₂-2-pyrimidinyl | 580 | |
| 38 | Cl | H | CF₃ | —C(═O)NH(CH₂)₂S(═O)₂CH₃ | 594 | |
| 39 | Cl | H | CF₃ | —C(═O)NHCH₂C(═O)NH₂ | 545 | |
| 40 | Cl | H | CF₃ | 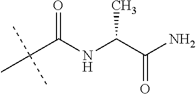 | 559 | |
| 41 | Cl | H | CF₃ | —C(═O)NH(CH₂)₂S(═O)CH₃ | 578 | |
| 42 | CF₃ | F | H | —C(═O)NH-t-Bu | 528 | |
| 43 | Cl | F | Cl | 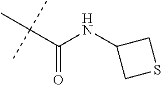 | 544 | |
| 44 | Cl | F | Cl | —C(═O)NH(CH₂)₂OCH₃ | 531 | |
| 45 | Cl | F | Cl | —C(═O)NHCH₂CH═CH₂ | 512 | |
| 46 | Cl | F | Cl | —C(═O)NHCH₂CH₃ | 500 | |
| 47 | Cl | F | Cl | —C(═O)NH-i-Bu | 528 | |
| 48 | Cl | F | Cl | —C(═O)NHCH₂C≡CH | 510 | |
| 49 | Cl | F | Cl | —C(═O)NHCH(CH₃)CN | 525 | |
| 50 | Cl | F | Cl | —C(═O)NHCH(CH₃)CH₂C≡N | 539 | |
| 51 | Cl | F | Cl | —C(═O)NH(CH₂)₂C≡N | 525 | |
| 52 | CF₃ | H | H | —C(═O)NHCH(CH₃)C≡N | 507 | |
| 53 | CF₃ | H | H | —C(═O)NHCH(c-Pr)C≡N | 533 | |
| 54 | CF₃ | H | H | —C(═O)NHCH(i-Pr)C≡N | 535 | |
| 55 | CF₃ | F | H | —C(═O)NH-c-Pr | 512 | |
| 56 | Cl | F | Cl | 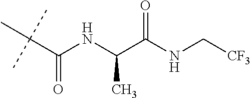 | | 150-154 |
| 57 | Cl | F | Cl | —C(═O)NHCH₂C(═O)NHCH₂CF₃ | | 219-223 |
| 58 | CF₃ | F | H | —C(═O)NHCH₂-c-Pr | 526 | |
| 59 | CF₃ | F | H | —C(═O)NH-i-Pr | 514 | |
| 60 | CF₃ | F | H | —C(═O)NHCH₂CH₃ | 500 | |
| 61 | Cl | F | Cl | —C(═O)NHCH(CH₃)-c-Pr | 540 | |
| 62 | Cl | F | Cl | 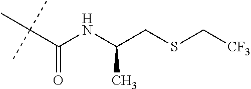 | | 126-130 |
| 63 | Cl | F | Cl | C(═O)NHCH(CH₃)CH₂S(═O)₂CH₃ | 592 | |
| 64 | Cl | F | Cl | 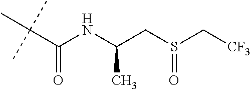 | | 271-275 |
| 65 | Cl | F | Cl | 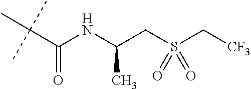 | | 147-151 |
| 66 | CF₃ | F | H | —C(═O)NH-n-Pr | 514 | |
| 67 | Cl | H | Cl | —C(═O)NHCH₂-2-pyrimidinyl | 546 | |
| 68 * | Cl | F | Cl | —C(═O)NHCH₂-2-pyrimidinyl | | 133-137 |
| 69 * | Cl | F | Cl | —C(═O)NHCH₂-2-pyrimidinyl | | 114-118 |
| 70 | Cl | H | Cl | —C(═O)NH-c-Pr | 494 | |
| 71 | CF₃ | F | H | —C(═O)NH-i-Bu | 528 | |
| 72 | CF₃ | F | H | —C(═O)NH(CH₂)₂OCH₃ | 530 | |
| 73 | CF₃ | F | H | —C(═O)NHCH₂CH═CH₂ | 512 | |
| 74 | CF₃ | F | H | —C(═O)NHCH₂C≡CH | 510 | |
| 75 | Cl | F | CF₃ | —C(═O)NHCH₂-2-pyrimidinyl | | 101-105 |
| 76 | CF₃ | F | H | —C(═O)NHCH₂-2-pyrimidinyl | | 109-113 |
| 77 | Cl | F | CF₃ | —C(═O)NHCH₂-c-Pr | | 198-202 |
| 78 | Cl | H | Cl | —C(═O)NHCH(CH₃)C≡N | | 139.5-160.7 |
| 79 | CF₃ | F | H | —C(═O)NHCH(CH₃)C≡N | 525 | |
| 80 | Cl | F | CF₃ | —C(═O)NH-c-Pr | | 147-151 |
| 81 | Cl | F | Cl | —C(═O)NHCH═NOCH₃ | 529 | |
| 82 | Cl | F | Cl | —C(═O)NH-c-Pr | 512 | 245-249 |
| [Note 1] | | | | | | |
| 83 | Cl | F | Cl | —C(═O)NH-c-Pr | 512 | 244-248 |
| [Note 2] | | | | | | |
| 84 | Cl | H | CF₃ | —C(═O)NHCH₃ | | 204-208 |
| 85 | Cl | H | CF₃ | —C(═O)NH-i-Pr | | 134-138 |
| 86 | Cl | H | CF₃ | —C(═O)NH-c-Bu | | 112-116 |
| 87 | CF₃ | H | H | —C(═O)NH-i-Pr | | 178-182 |
| 88 | CF₃ | H | H | —C(═O)NH-c-Bu | | 120-124 |
| 89 | Cl | H | CF₃ | —C(═O)NH-n-Pr | | 92-96 |
| 90 | Cl | H | CF₃ | —C(═O)NHCH₂CH₃ | | 106-110 |
| 91 | Cl | H | CF₃ | —C(═O)NHCH₂CH═CH₂ | | 94-98 |
| 92 | Cl | H | CF₃ | —C(═O)NHCH₂C≡CH | | 111-115 |
| 93 | CF₃ | H | H | —C(═O)NHCH₃ | | 125-130 |
| 94 | CF₃ | H | H | —C(═O)NHCH₂CH═CH₂ | | 122-126 |
| 95 | Cl | H | CF₃ | —C(═O)NH-i-Bu | 183-187 | |
| 96 | CF₃ | H | H | —C(═O)NHCH₂CH₃ | | 200-204 |
| 97 | CF₃ | H | H | —C(═O)NH-n-Pr | | 120-124 |
| 98 | CF₃ | H | H | —C(═O)NHCH₂-c-Pr | | 171-175 |
| [Note 3] | | | | | | |
| 99 | CF₃ | H | H | —C(═O)NHCH₂-c-Pr | | 171-175 |
| [Note 4] | | | | | | |
| 100 | CF₃ | H | H | —C(═O)NH-i-Bu | | 94-98 |
| 101 | CF₃ | H | H | —C(═O)NHCH₂C≡CH | | 183-187 |

* Single enantiomer at the 5 position of the isoxazoline ring.
Note 1:
Enantiomer (99.70% ee) at the 5 position of the isoxazoline ring [α]_{D}²⁰ −22.6280° (concentration = 0.5% in chloroform).
Note 2:
Enantiomer (99.90% ee) at the 5 position of the isoxazoline ring [α]_{D}²⁰ +15.4840° (concentration = 0.5% in chloroform).
Note 3:
Enantiomer (99.75% ee) at the 5 position of the isoxazoline ring [α]_{D}²⁰ −6.620° (concentration = 0.1% in chloroform).
Note 4:
Enantiomer (97.27% ee) at the 5 position of the isoxazoline ring [α]_{D}²⁰ +20.540° (concentration = 0.1% in chloroform).

A compound of this disclosure will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil in water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil in water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethylphosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters alkyl and aryl benzoates, γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present disclosure often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this disclosure may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all formulations are prepared in conventional ways. Compound numbers refer to compounds in Index Tables A-B. Without further elaboration, it is believed that one skilled in the art using the preceding description can utilize the present disclosure to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 2 | 98.5% |
| silica aerogel | 0.5% |
| synthetic amorphous fine silica | 1.0% |

### Example B

Wettable Powder

| | |
|---|---|
| Compound 3 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 4 | 10.0% |
| attapulgite granules | 90.0% |
| (low volatile matter, | |
| 0.71/0.30 mm; | |
| U.S.S. No. 25-50 sieves) | |

### Example D

Extruded Pellet

| | |
|---|---|
| Compound 23 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 2 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 3 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 4 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 23 | 2.5% |
| pyrrolidone-styrene copolymer | 4.8% |
| tristyrylphenyl 16-ethoxylate | 2.3% |
| talc | 0.8% |
| corn starch | 5.0% |
| slow-release fertilizer | 36.0% |
| kaolin | 38.0% |
| water | 10.6% |

### Example I

Suspension Concentrate

| | |
|---|---|
| compound 2 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| compound 3 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| compound 4 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 23 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Compounds of this disclosure exhibit activity against a wide spectrum of invertebrate pests. These pests include invertebrates inhabiting a variety of environments such as, for example, plant foliage, roots, soil, harvested crops or other foodstuffs, or building structures. These pests include, for example, invertebrates feeding on foliage (including leaves, stems, flowers and fruits), seeds, wood or textile fibers, and thereby causing injury or damage to, for example, growing or stored agronomic crops, forests, greenhouse crops, ornamentals, nursery crops, stored foodstuffs or fiber products, or houses or other structures or their contents. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, INVICTA RR2 PRO™, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may exhibit enhanced effects with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may exhibit enhance effects with the phenotypic expression of proteins or other natural products toxic to invertebrate pests to provide greater-than-additive control of these pests.

Compositions of this disclosure can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present disclosure which further comprise at least one plant nutrient can be in the form of liquids or solids. Of note are solid formulations in the form of granules, small sticks or tablets. Solid formulations comprising a fertilizer composition can be prepared by mixing the compound or composition of the present disclosure with the fertilizer composition together with formulating ingredients and then preparing the formulation by methods such as granulation or extrusion. Alternatively solid formulations can be prepared by spraying a solution or suspension of a compound or composition of the present disclosure in a volatile solvent onto a previous prepared fertilizer composition in the form of dimensionally stable mixtures, e.g., granules, small sticks or tablets, and then evaporating the solvent.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera frugiperda J. E. Smith), beet armyworm (Spodoptera exigua Hubner), cotton leafworm (Spodoptera littoralis Boisduval), yellowstriped armyworm (Spodoptera ornithogalli Guenée), black cutworm (Agrotis Ipsilon Hufnagel), velvetbean caterpillar (Anticarsia gemmatalis Hubner), green fruitworm (Lithophane antennata Walker), cabbage armyworm (Barathra brassicae Linnaeus), soybean looper (Pseudoplusia includens Walker), cabbage looper (Trichoplusia ni Hubner), tobacco budworm (Hehothis virescens Fabricius)); borers, casebearers, webworms, coneworms, cabbageworms and skeletonizers from the family Pyralidae (e.g., European corn borer (Ostrinia nubilalis Hubner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guenée), green leafroller (Cnaphalocrocis medinalis), grape leaffolder (Desmia funeralis Hubner), melon worm (Diaphania nitidalis Stoll), cabbage center grub (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), striped riceborer (Chilo suppressalis Walker), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hubner), apple bud moth (Pandemis pyrusana Kearfott), omnivorous leafroller (Platynota stultana Walsingham), barred fruit-tree tortrix (Pandemis cerasana Hubner), apple brown tortrix (Pandemis heparana Denis & Schiffermüller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites, dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Förster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard). Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of the disclosure may have activity against pests in the order Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hubner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermüller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hubner (cabbage looper) and Tuta absoluta Meyrick (tomato leafminer)).

Compounds of the disclosure have significant activity on members from the order Homoptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stal (rice leafhopper), Nilaparvata lugens Stal (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this disclosure also have activity on members from the order Hemiptera including: Acrosternum hilare Say (green stink bug), Anasa tristis De Geer (squash bug), Blissus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schäffer (cotton stainer), Euchistus servus Say (brown stink bug), Euchistus variolarius Palisot de Beauvois (one-spotted stink bug), Graptosthetus spp. (complex of seed bugs), Halymorpha halys Stål (brown marmorated stink bug), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the disclosure include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Note that some contemporary classification systems place Homoptera as a suborder within the order Hemiptera.

Of note is use of compounds of this disclosure for controlling diamondback moth (Plutella xylostella). Of note is use of compounds of this disclosure for controlling fall armyworm (Spodoptera frugiperda). Of note is use of compounds of this disclosure for controlling western flower thrips (Frankliniella occidentalis). Of note is use of compounds of this disclosure for controlling potato leafhopper (Empoasca fabae). Of note is use of compounds of this disclosure for controlling cotton melon aphid (Aphis gossypii). Of note is use of compounds of this disclosure for controlling green peach aphid (Myzus persicae). Of note is use of compounds of this disclosure for controlling sweetpotato whitefly (Bemisia tabaci).

Compounds of the present disclosure may also be useful for increasing vigor of a crop plant. This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present disclosure may increase the vigor of treated plants compared to untreated plants by killing or otherwise preventing feeding of phytophagous invertebrate pests in the environment of the plants. In the absence of such control of phytophagous invertebrate pests, the pests reduce plant vigor by consuming plant tissues or sap, or transmiting plant pathogens such as viruses. Even in the absence of phytophagous invertebrate pests, the compounds of the disclosure may increase plant vigor by modifying metabolism of plants. Generally, the vigor of a crop plant will be most significantly increased by treating the plant with a compound of the disclosure if the plant is grown in a nonideal environment, i.e. an environment comprising one or more aspects adverse to the plant achieving the full genetic potential it would exhibit in an ideal environment.

Of note is a method for increasing vigor of a crop plant wherein the crop plant is grown in an environment comprising phytophagous invertebrate pests. Also of note is a method for increasing vigor of a crop plant wherein the crop plant is grown in an environment not comprising phytophagous invertebrate pests. Also of note is a method for increasing vigor of a crop plant wherein the crop plant is grown in an environment comprising an amount of moisture less than ideal for supporting growth of the crop plant. Of note is a method for increasing vigor of a crop plant wherein the crop is rice. Also of note is a method for increasing vigor of a crop plant wherein the crop is maize (corn). Also of note is a method for increasing vigor of a crop plant wherein the crop is soybean.

Compounds of this disclosure can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus the present disclosure also pertains to a composition comprising a biologically effective amount of a compound of Formula 1, at least one additional component selected from the group consisting of surfactants, solid diluents and liquid diluents, and at least one additional biologically active compound or agent. For mixtures of the present disclosure, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this disclosure can be formulated are insecticides such as abamectin, acephate, acequinocyl, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, bifenthrin, bifenazate, bistrifluron, borate, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)-1H-pyrazole-5-carboxamide), cycloprothrin, cycloxaprid ((5S,8R)-1-[(6-chloro-3 pyridinyl)methyl]2,3,5,6,7,8-hexahydro-9-nitro-5,8-Epoxy-1H-imidao[1,2-a]azepine) cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, flubendiamide, flucythrinate, flufenerim, flufenoxuron, flufenoxystrobin (methyl (αE)-2-[[2-chloro-4-(trifluoromethyl)phenoxy]methyl]-α-(methoxymethylene)benzeneacetate), flufensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), fluvalinate, tau-fluvalinate, fonophos, formetanate, fosthiazate, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiodicarb, methomyl, methoprene, methoxychlor, metofluthrin, methoxyfenozide, metofluthrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spirotetramat, sulprofos, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, terbufos, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, Bacillus thuringiensis delta-endotoxins, entomopathogenic bacteria, entomopathogenic viruses and entomopathogenic fungi.

Of note are insecticides such as abamectin, acetamiprid, acrinathrin, afidopyropen, amitraz, avermectin, azadirachtin, benfuracarb, bensultap, bifenthrin, buprofezin, cadusafos, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, flufensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nucleo polyhedrosis viruses.

One embodiment of biological agents for mixing with compounds of this disclosure include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Ind., USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydia pomonella granulosis virus (CpGV).

Of particular note is such a combination where the other invertebrate pest control active ingredient belongs to a different chemical class or has a different site of action than the compound of Formula 1. In certain instances, a combination with at least one other invertebrate pest control active ingredient having a similar spectrum of control but a different site of action will be particularly advantageous for resistance management. Thus, a composition of the present disclosure can further comprise a biologically effective amount of at least one additional invertebrate pest control active ingredient having a similar spectrum of control but belonging to a different chemical class or having a different site of action. These additional biologically active compounds or agents include, but are not limited to, acetylcholinesterase (AChE) inhibitors such as the carbamates methomyl, oxamyl, thiodicarb, triazamate, and the organophosphates chlorpyrifos; GABA-gated chloride channel antagonists such as the cyclodienes dieldrin and endosulfan, and the phenylpyrazoles ethiprole and fipronil; sodium channel modulators such as the pyrethroids bifenthrin, cyfluthrin, beta-cyfluthrin, cyhalothrin, lambda-cyhalothrin, cypermethrin, deltamethrin, dimefluthrin, esfenvalerate, metofluthrin and profluthrin; nicotinic acetylcholinereceptor (nAChR) agonists such as the neonicotinoids acetamiprid, clothianidin, dinotefuran, imidacloprid, nitenpyram, nithiazine, thiacloprid, and thiamethoxam, and sulfoxaflor; nicotinic acetylcholine receptor (nAChR) allosteric activators such as the spinosyns spinetoram and spinosad; chloride channel activators such as the avermectins abamectin and emamectin; juvenile hormone mimics such as diofenolan, methoprene, fenoxycarb and pyriproxyfen; selective homopteran feeding blockers such as pymetrozine and flonicamid; mite growth inhibitors such as etoxazole; inhibitors of mitochondrial ATP synthase such as propargite; ucouplers of oxidative phosphorylation via disruption of the proton gradient such as chlorfenapyr; nicotinic acetylcholine receptor (nAChR) channel blockers such as the nereistoxin analogs cartap; inhibitors of chitin biosynthesis such as the benzoylureas flufenoxuron, hexaflumuron, lufenuron, novaluron, noviflumuron and triflumuron, and buprofezin; dipteran moulting disrupters such as cyromazine; ecdysone receptor agonists such as the diacylhydrazines methoxyfenozide and tebufenozide; octopamine receptor agonists such as amitraz; mitochondrial complex III electron transport inhibitors such as hydramethylnon; mitochondrial complex I electron transport inhibitors such as pyridaben; voltage-dependent sodium channel blockers such as indoxacarb; inhibitors of acetyl CoA carboxylase such as the tetronic and tetramic acids spirodiclofen, spiromesifen and spirotetramat; mitochondrial complex II electron transport inhibitors such as the ß-ketonitriles cyenopyrafen and cyflumetofen; ryanidine receptor modulators such as the anthranilic diamides chlorantraniliprole, cyantraniliprole and cyantraniliprole, diamides such as flubendiamide, and ryanodine receptor ligands such as ryanodine; compounds wherein the target site responsible for biological activity is unknown or uncharacterized such as azadirachtin, bifenazate, pyridalyl, pyrifluquinazon and triflumezopyrim; microbial disrupters of insect midgut membranes such as Bacillus thuringensis and the delta-endotoxins they produce and Bacillus sphaericus; and biological agents including nucleo polyhedro viruses (NPV) and other naturally occurring or genetically modified insecticidal viruses.

Further examples of biologically active compounds or agents with which compounds of this disclosure can be formulated are: fungicides such as acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, di ethofencarb, difenoconazole, diflumetorim, dimethirimol, dimethomorph, dimoxystrobin, diniconazole (including diniconazole-M), dinocap, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpropidin, fenpropimorph, fenpyrazamine, fentin acetate, fentin hydroxide, ferbam, ferimzone, flometoquin, fluazinam, fludioxonil, flufenoxystrobin, flumorph, fluopicolide, fluopyram, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, iodicarb, ipconazole, isofetamid, iprobenfos, iprodione, iprovalicarb, isoprothiolane, isopyrazam, isotianil, kasugamycin, kresoxim-methyl, mancozeb, mandipropamid, mandestrobin, maneb, mapanipyrin, mepronil, meptyldinocap, metalaxyl (including metalaxyl-M/mefenoxam), metconazole, methasulfocarb, metiram, metominostrobin, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pyraclostrobin, pyrametostrobin, pyraoxystrobin, pyrazophos, pyribencarb, pyributacarb, pyrifenox, pyriofenone, perisoxazole, pyrimethanil, pyrifenox, pyrrolnitrin, pyroquilon, quinconazole, quinmethionate, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide and 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone; nematocides such as fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus and Pasteuria nishizawae; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

In certain instances, combinations of a compound of this disclosure with other biologically active (particularly invertebrate pest control) compounds or agents (i.e. active ingredients) can result in an enhanced effect. Reducing the quantity of active ingredients released in the environment while ensuring effective pest control is always desirable. When enhanced invertebrate pest control occurs at application rates giving agronomically satisfactory levels of invertebrate pest control, such combinations can be advantageous for reducing crop production cost and decreasing environmental load.

Compounds of this disclosure and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The exogenously applied invertebrate pest control compounds of this disclosure in combination with the expressed toxin proteins may provide an enhanced effect.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001.

Invertebrate pests are controlled in agronomic and nonagronomic applications by applying one or more compounds of this disclosure, typically in the form of a composition, in a biologically effective amount, to the environment of the pests, including the agronomic and/or nonagronomic locus of infestation, to the area to be protected, or directly on the pests to be controlled.

Thus the present disclosure comprises a method for controlling an invertebrate pest in agronomic and/or nonagronomic applications, comprising contacting the invertebrate pest or its environment with a biologically effective amount of one or more of the compounds of the disclosure, or with a composition comprising at least one such compound or a composition comprising at least one such compound and a biologically effective amount of at least one additional biologically active compound or agent. Examples of suitable compositions comprising a compound of the disclosure and a biologically effective amount of at least one additional biologically active compound or agent include granular compositions wherein the additional active compound is present on the same granule as the compound of the disclosure or on granules separate from those of the compound of the disclosure.

To achieve contact with a compound or composition of the disclosure to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the disclosure can be applied to the plant foliage or the soil. Compounds of this disclosure can also be effectively delivered through plant uptake by contacting the plant with a composition comprising a compound of this disclosure applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a compound of the present disclosure or with a composition comprising a biologically effective amount of a compound of the present disclosure. Of further note is this method wherein the environment is soil and the composition is applied to the soil as a soil drench formulation. Of further note is that compounds of this disclosure are also effective by localized application to the locus of infestation. Other methods of contact include application of a compound or a composition of the disclosure by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the disclosure. The compounds of this disclosure can also be impregnated into materials for fabricating invertebrate control devices (e.g., insect netting).

Compounds of the disclosure are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant and seed cultivars which can be treated according to the disclosure include those that are resistant against one or more biotic stresses (pests such as nematodes, insects, mites, fungi, etc.) or abiotic stresses (drought, cold temperature, soil salinity, etc.), or that contain other desirable characteristics. Plants and seeds can be genetically modified to exhibit traits of, for example, herbicide tolerance, insect-resistance, modified oil profiles or drought tolerance.

Treatment of genetically modified plants and seeds with compounds of the disclosure may result in super-additive or enhanced effects. For example, reduction in application rates, broadening of the activity spectrum, increased tolerance to biotic/abiotic stresses or enhanced storage stability may be greater than expected from just simple additive effects of the application of compounds of the disclosure on genetically modified plants and seeds.

Compounds of this disclosure are also useful in seed treatments for protecting seeds from invertebrate pests. In the context of the present disclosure and claims, treating a seed means contacting the seed with a biologically effective amount of a compound of this disclosure, which is typically formulated as a composition of the disclosure. This seed treatment protects the seed from invertebrate soil pests and generally can also protect roots and other plant parts in contact with the soil of the seedling developing from the germinating seed. The seed treatment may also provide protection of foliage by translocation of the compound of this disclosure or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this disclosure can also increase vigor of plants growing from the seed.

One method of seed treatment is by spraying or dusting the seed with a compound of the disclosure (i.e. as a formulated composition) before sowing the seeds. Compositions formulated for seed treatment generally comprise a film former or adhesive agent. Therefore typically a seed coating composition of the present disclosure comprises a biologically effective amount of a compound of Formula 1, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 and their compositions, both alone and in combination with other insecticides, nematicides, and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides with which compounds of Formula 1 can be formulated to provide mixtures useful in seed treatment include abamectin, acetamiprid, acrinathrin, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of nucleo polyhedrosis viruses.

Fungicides with which compounds of Formula 1 can be formulated to provide mixtures useful in seed treatment include amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM 1-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The treated seed typically comprises a compound of the present disclosure in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this disclosure can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

One embodiment of the present disclosure relates to a method for controlling invertebrate pests, comprising diluting the pesticidal composition of the present disclosure (a compound of Formula 1 formulated with surfactants, solid diluents and liquid diluents or a formulated mixture of a compound of Formula 1 and at least one other pesticide) with water, and optionally adding an adjuvant to form a diluted composition, and contacting the invertebrate pest or its environment with an effective amount of said diluted composition.

Although a spray composition formed by diluting with water a sufficient concentration of the present pesticidal composition can provide sufficient efficacy for controlling invertebrate pests, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

Among the spray adjuvants, oils including crop oils, crop oil concentrates, vegetable oil concentrates and methylated seed oil concentrates are most commonly used to improve the efficacy of pesticides, possibly by means of promoting more even and uniform spray deposits. In situations where phytotoxicity potentially caused by oils or other water-immiscible liquids are of concern, spray compositions prepared from the composition of the present disclosure will generally not contain oil-based spray adjuvants. However, in situations where phytotoxicity caused by oil-based spray adjuvants is commercially insignificant, spray compositions prepared from the composition of the present composition can also contain oil-based spray adjuvants, which can potentially further increase control of invertebrate pests, as well as rainfastness.

Products identified as “crop oil” typically contain 95 to 98% paraffin or naphtha-based petroleum oil and 1 to 2% of one or more surfactants functioning as emulsifiers. Products identified as “crop oil concentrates” typically consist of 80 to 85% of emulsifiable petroleum-based oil and 15 to 20% of nonionic surfactants. Products correctly identified as “vegetable oil concentrates” typically consist of 80 to 85% of vegetable oil (i.e. seed or fruit oil, most commonly from cotton, linseed, soybean or sunflower) and 15 to 20% of nonionic surfactants. Adjuvant performance can be improved by replacing the vegetable oil with methyl esters of fatty acids that are typically derived from vegetable oils. Examples of methylated seed oil concentrates include MSO® Concentrate (UAP-Loveland Products, Inc.) and Premium MSO Methylated Spray Oil (Helena Chemical Company).

The amount of adjuvants added to spray mixtures generally does not exceed about 2.5% by volume, and more typically the amount is from about 0.1 to about 1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

The compounds of this disclosure can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present disclosure. Combinations with spray oils, spray oil concentrations, spreader stickers, adjuvants, other solvents, and piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present disclosure and a carrier. One embodiment of such a spray composition comprises a biologically effective amount of a compound or a composition of the present disclosure and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

The following Tests demonstrate the control efficacy of compounds of this disclosure on specific pests. “Control efficacy” represents inhibition of invertebrate pest development (including mortality) that causes significantly reduced feeding. The pest control protection afforded by the compounds is not limited, however, to these species. See Index Table A for compound descriptions.

### Biological Examples of the Invention

Formulation and Spray Methodology for Tests A-H

Test compounds were formulated using a solution containing 10% acetone, 90% water and 300 ppm Activator 90® non-ionic surfactant (Loveland Products, Loveland, Colo., USA). The formulated compounds were applied in 1 mL of liquid through an atomizer nozzle positioned 1.27 cm (0.5 inches) above the top of each test unit. Test compounds were sprayed at the rates indicated, and each test was replicated three times.

### Test A

For evaluating control of diamondback moth (Plutella xylostella (L.)) the test unit consisted of a small open container with a 12-14-day-old mustard plant inside. This was pre-infested with ˜50 neonate larvae that were dispensed into the test unit via corn cob grits using an inoculator. The larvae moved onto the test plant after being dispensed into the test unit.

Test compounds were formulated and sprayed at 250, 50, 10, 2, and 0.4 ppm. After spraying of the formulated test compound, each test unit was allowed to dry for 1 hour and then a black, screened cap was placed on top. The test units were held for 6 days in a growth chamber at 25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (40% or less feeding damage and/or 100% mortality): 1 and 2.

Of the compounds of Formula 1 tested at 50 ppm, the following provided very good to excellent levels of control efficacy (40% or less feeding damage and/or 100% mortality): 1 and 2.

Of the compounds of Formula 1 tested at 10 ppm, the following provided very good to excellent levels of control efficacy (40% or less feeding damage and/or 100% mortality): 1, 2, 3, 4, 5, 6, 7, 8, 9, 10, 11, 12, 13, 14, 15, 16, 17, 18, 19, 20, 21, 22, 23, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 59, 70, 71, and 74.

Of the compounds of Formula 1 tested at 2 ppm, the following provided very good to excellent levels of control efficacy (40% or less feeding damage and/or 100% mortality): 1, 2, 3, 4, 5, 6, 7, 8, 9, 10, 11, 12, 13, 14, 15, 16, 17, 18, 19, 20, 21, 23, 26, 27, 28, 29, 30, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 64, 65, 66, 67, 68, 70, 71, 72, 73, 74, 75, 76, 77, 78, 79, 80, 81, 82, 84, 85, 86, 87, 88, 89, 90, 91, 92, 93, 94, 95, and 96.

Of the compounds of Formula 1 tested at 0.4 ppm, the following provided very good to excellent levels of control efficacy (40% or less feeding damage and/or 100% mortality): 3, 10, 11, 13, 15, 16, 17, 18, 20, 23, 26, 27, 28, 30, 32, 33, 35, 36, 37, 42, 43, 44, 45, 46, 47, 48, 49, 50, 51, 52, 53, 54, 55, 56, 58, 59, 60, 61, 66, 67, 68, 70, 71, 72, 73, 74, 75, 76, 77, 78, 79, 80, 81, 82, 84, 85, 86, 87, 88, 89, 90, 91, 92, 94, 95, and 96.

### Test B

For evaluating control of fall armyworm (Spodoptera frugiperda (J. E. Smith)) the test unit consisted of a small open container with a 4-5-day-old corn (maize) plant inside. This was pre-infested with 10-15 1-day-old larvae on a piece of insect diet.

Test compounds were formulated and sprayed at 250, 50, 10, 2, and 0.4 ppm. After spraying of the formulated test compound, the test units were maintained in a growth chamber for 6 days at 25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (40% or less feeding damage and/or 100% mortality): 1 and 2.

Of the compounds of Formula 1 tested at 50 ppm, the following provided very good to excellent levels of control efficacy (40% or less feeding damage and/or 100% mortality): 1 and 2.

Of the compounds of Formula 1 tested at 10 ppm, the following provided very good to excellent levels of control efficacy (40% or less feeding damage and/or 100% mortality): 2, 3, 4, 6, 7, 8, 9, 10, 11, 13, 15, 16, 17, 18, 19, 20, 23, 26, 27, 28, 29, 32, 33, 34, 35, 36, 37, 38, 42, 44, 45, 46, 47, 48, 49, 50, 51, 52, 53, 54, 55, 59, 70, 71, and 74.

Of the compounds of Formula 1 tested at 2 ppm, the following provided very good to excellent levels of control efficacy (40% or less feeding damage and/or 100% mortality): 2, 3, 4, 10, 13, 16, 17, 18, 20, 23, 28, 32, 33, 35, 36, 37, 44, 45, 46, 47, 48, 53, 55, 58, 59, 60, 61, 66, 67, 68, 70, 71, 72, 73, 74, 75, 77, 78, 80, 81, 82, 84, 85, 86, 87, 88, 89, 90, 91, 92, 94, 95, 96, and 101.

Of the compounds of Formula 1 tested at 0.4 ppm, the following provided very good to excellent levels of control efficacy (40% or less feeding damage and/or 100% mortality): 3, 13, 16, 17, 23, 28, 35, 36, 37, 47, 48, 58, 59, 60, 61, 66, 68, 70, 73, 74, 77, 81, 82, 84, 85, 88, 89, 90, 91, and 92.

### Test C

For evaluating control of corn planthopper (Peregrinus maidis (Ashmead)) through contact and/or systemic means, the test unit consisted of a small open container with a 3-4-day-old corn (maize) plant inside. White sand was added to the top of the soil prior to application of the test compound.

Test compounds were formulated and sprayed at 50 and 10 ppm. After spraying of the formulated test compound, the test units were allowed to dry for 1 h before they were post-infested with ˜15-20 nymphs (18-to-21-day-old). A black, screened cap was placed on the top of each test unit, and the test units were held for 6 days in a growth chamber at 22-24° C. and 50-70% relative humidity. Each test unit was then visually assessed for insect mortality.

Of the compounds of Formula 1 tested at 50 ppm, the following resulted in at least 80% mortality: 4, 6, 18, 29, 35, 37, 48, 55, 58, 66, 68, 72, 73, 74, 75, 76, 80, 81, 86, 88, 90, and 92.

Of the compounds of Formula 1 tested at 10 ppm, the following resulted in at least 80% mortality: 76.

### Test D

For evaluating control of potato leafhopper (Empoasca fabae (Harris)) through contact and/or systemic means, the test unit consisted of a small open container with a 5-6-day-old Soleil bean plant (primary leaves emerged) inside. White sand was added to the top of the soil, and one of the primary leaves was excised prior to application of the test compound.

Test compounds were formulated and sprayed at 250, 50 and 10 ppm. After spraying of the formulated test compound, the test units were allowed to dry for 1 hour before they were post-infested with 5 potato leafhoppers (18-to-21-day-old adults). A black, screened cap was placed on the top of the test unit, and the test units were held for 6 days in a growth chamber at 20° C. and 70% relative humidity. Each test unit was then visually assessed for insect mortality.

Of the compounds of Formula 1 tested at 250 ppm, the following resulted in at least 80% mortality: 2.

Of the compounds of Formula 1 tested at 50 ppm, the following resulted in at least 80% mortality: 2, 4, 6, 7, 10, 11, 13, 14, 20, 21, 23, 28, 32, 35, 36, 37, 43, 45, 48, 49, 53, 55, 58, 61, 62, 66, 68, 70, 71, 74, 76, 77, 80, 81, 82, 86, and 92.

Of the compounds of Formula 1 tested at 10 ppm, the following resulted in at least 80% mortality: 11, 13, 35, 36, 61, and 81.

### Test E

For evaluating control of green peach aphid (Myzus persicae (Sulzer)) through contact and/or systemic means, the test unit consisted of a small open container with a 12-15-day-old radish plant inside. This was pre-infested by placing on a leaf of the test plant 30-40 aphids on a piece of leaf excised from a culture plant (cut-leaf method). The aphids moved onto the test plant as the leaf piece desiccated. After pre-infestation, the soil of the test unit was covered with a layer of sand.

Test compounds were formulated and sprayed at 250, 50 and 10 ppm. After spraying of the formulated test compound, each test unit was allowed to dry for 1 hour and then a black, screened cap was placed on top. The test units were held for 6 days in a growth chamber at 19-21° C. and 50-70% relative humidity. Each test unit was then visually assessed for insect mortality.

Of the compounds of Formula 1 tested at 250 ppm, the following resulted in at least 80% mortality: 1 and 2.

Of the compounds of Formula 1 tested at 50 ppm, the following resulted in at least 80% mortality: 2, 3, 4, 6, 7, 10, 11, 13, 16, 17, 18, 20, 23, 27, 28, 29, 32, 34, 35, 36, 37, 38, 45, 48, 53, 55, 56, 58, 60, 61, 66, 67, 68, 70, 71, 72, 73, 74, 75, 76, 77, 80, 81, 82, 85, 86, 87, 88, 89, 90, 91, and 92.

Of the compounds of Formula 1 tested at 10 ppm, the following resulted in at least 80% mortality: 3, 6, 13, 17, 18, 20, 23, 28, 35, 36, 45, 58, 61, 66, 68, 81, 82, 86, 89, and 90.

### Test F

For evaluating control of cotton melon aphid (Aphis gossypii (Glover)) through contact and/or systemic means, the test unit consisted of a small open container with a 5-day-old okra plant inside. This was pre-infested with 30-40 insects on a piece of leaf according to the cut-leaf method, and the soil of the test unit was covered with a layer of sand.

Test compounds were formulated and sprayed at 250, 50 and 10 ppm. After spraying, the test units were maintained in a growth chamber for 6 days at 19° C. and 70% relative humidity. Each test unit was then visually assessed for insect mortality.

Of the compounds of Formula 1 tested at 250 ppm, the following resulted in at least 80% mortality: 1 and 2.

Of the compounds of Formula 1 tested at 50 ppm, the following resulted in at least 80% mortality: 1, 2, 3, 4, 6, 7, 8, 10, 11, 12, 13, 15, 17, 18, 19, 20, 21, 23, 25, 26, 27, 28, 29, 30, 32, 35, 36, 37, 40, 43, 44, 45, 46, 48, 53, 55, 56, 58, 59, 60, 61, 62, 66, 67, 68, 70, 71, 72, 73, 74, 75, 76, 77, 79, 80, 81, 82, 84, 85, 86, 87, 89, 90, 91, 92, 94, 96, and 101.

Of the compounds of Formula 1 tested at 10 ppm, the following resulted in at least 80% mortality: 2, 3, 4, 6, 13, 17, 20, 23, 28, 35, 36, 37, 46, 55, 58, 61, 66, 67, 68, 70, 72, 73, 76, 80, 81, 82, 85, 86, 89, and 90.

### Test G

For evaluating control of the sweetpotato whitefly (Bemisia tabaci (Gennadius)) through contact and/or systemic means, the test unit consisted of a small open container with a 12-14-day-old cotton plant inside. Prior to the spray application, both cotyledons were removed from the plant, leaving one true leaf for the assay. Adult whiteflies were allowed to lay eggs on the plant and then were removed from the test unit. Cotton plants infested with at least 15 eggs were submitted to the test for spraying.

Test compounds were formulated and sprayed at 250, 50 and 10 ppm. After spraying, the test units were allowed to dry for 1 hour. The cylinders were then removed, and the units were taken to a growth chamber and held for 13 days at 28° C. and 50-70% relative humidity. Each test unit was then visually assessed for insect mortality.

Of the compounds of Formula 1 tested at 250 ppm, the following resulted in at least 50% mortality: 1 and 2.

Of the compounds of Formula 1 tested at 50 ppm, the following resulted in at least 50% mortality: 1, 2, 3, 4, 6, 7, 10, 13, 19, 20, 21, 26, 28, 32, 35, 36, 37, 44, 47, 53, 55, 56, 58, 61, 62, 66, 68, 71, 72, 74, 75, 76, 77, 79, and 80.

Of the compounds of Formula 1 tested at 10 ppm, the following resulted in at least 50% mortality: 2, 3, 4, 10, 13, 20, 28, 32, 35, 36, 37, 44, 47, 53, 55, 61, 71, and 80.

### Test H

For evaluating control of the Western Flower Thrips (Frankliniellla occidentalis (Pergande)) through contact and/or systemic means, the test unit consisted of a small open container with a 8-9-day-old Soleil bean plant inside.

Test compounds were formulated and sprayed at 250, 50, 10 and 2 ppm. After spraying, the test units were allowed to dry for 1 hour, and then ˜60 thrips (adults and nymphs) were added to each unit. A black, screened cap was placed on top, and the test units were held for 6 days at 25° C. and 45-55% relative humidity. Each test unit was then visually assessed for plant damage and insect mortality.

Of the compounds of Formula 1 tested at 250 ppm, the following provided very good to excellent levels of control efficacy (30% or less plant damage and/or 100% mortality): 1 and 2.

Of the compounds of Formula 1 tested at 50 ppm, the following provided very good to excellent levels of control efficacy (30% or less plant damage and/or 100% mortality): 1.

Of the compounds of Formula 1 tested at 10 ppm, the following provided very good to excellent levels of control efficacy (30% or less plant damage and/or 100% mortality): 2, 3, 4, 5, 6, 7, 8, 9, 10, 11, 12, 13, 14, 15, 16, 17, 18, 19, 20, 22, 23, 24, 25, 26, 27, 29, 30, 32, 33, 34, 35, 36, 37, 38, 39, 40, 41, 43, 44, 45, 46, 47, 48, 49, 50, 51, 52, 53, 54, 55, 59, 71, and 74.

Of the compounds of Formula 1 tested at 2 ppm, the following provided very good to excellent levels of control efficacy (30% or less plant damage and/or 100% mortality): 2, 3, 4, 5, 6, 7, 8, 9, 10, 11, 12, 13, 15, 16, 17, 18, 19, 20, 23, 26, 27, 28, 29, 30, 32, 33, 34, 35, 36, 37, 38, 41, 44, 45, 46, 47, 48, 49, 50, 51, 52, 53, 55, 58, 59, 60, 61, 66, 67, 68, 70, 71, 72, 73, 74, 75, 76, 77, 78, 79, 80, 81, 82, 84, 85, 86, 87, 88, 89, 90, 91, 92, 93, 94, 95, 96, and 101.

Of the compounds of Formula 1 tested at 0.4 ppm, the following provided very good to excellent levels of control efficacy (30% or less plant damage and/or 100% mortality): 2, 3, 4, 13, 16, 17, 23, 28, 35, 36, 37, 44, 45, 46, 47, 48, 53, 55, 58, 60, 61, 67, 68, 70, 74, 75, 76, 77, 78, 79, 80, 81, 82, 84, 85, 88, 89, 90, 91, 92, 93, 94, 95, 96, and 101.

What is claimed is:

1. A compound selected from Formula 1, 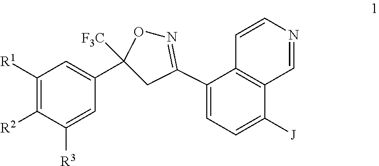
wherein
J is 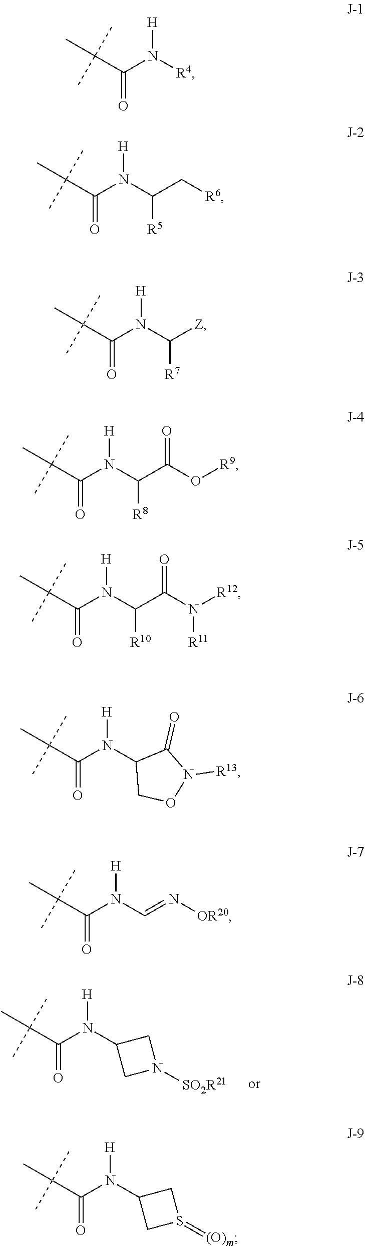
R¹ is H, Cl or CF₃;
R² is H, F or Cl;
R³ is H, Cl or CF₃;
R⁴ is C₁-C₆ alkyl, C₂-C₆ alkenyl, C₂-C₆ alkynyl, C₃-C₆ cycloalkyl, C₃-C₆ cycloalkenyl, C₄-C₈ alkylcycloalkyl or C₄-C₈ cycloalkylalkyl, each unsubstituted or substituted with substituents independently selected from halogen, cyano and CO₂R¹⁸;
R⁵ is H or C₁-C₄ alkyl;
R⁶ is OR¹⁴ or S(O)ₙR¹⁵;
R⁷ is H or C₁-C₄ alkyl;
R⁸ is H or C₁-C₄ alkyl;
R⁹ is H; or C₁-C₄ alkyl, unsubstituted or substituted with substituents independently selected from halogen, cyano, OR¹⁶, S(O)ₙR¹⁷ and CO₂R¹⁸;
R¹⁰ is H or C₁-C₄ alkyl;
R¹¹ is H or C₁-C₄ alkyl;
R¹² is H; or C₁-C₄ alkyl, unsubstituted or substituted with substituents independently selected from halogen, cyano, OR¹⁶, S(O)ₙR¹⁷ and CO₂R¹⁸;
R¹³ is H, C₁-C₄ alkyl or C₁-C₄ haloalkyl;
R¹⁴ is C₁-C₄ alkyl;
R¹⁵ is H, C₁-C₄ alkyl or C₁-C₄ haloalkyl;
each R¹⁶ is independently C₁-C₄ alkyl or C₁-C₄ haloalkyl;
each R¹⁷ is independently C₁-C₄ alkyl or C₁-C₄ haloalkyl;
each R¹⁸ is independently C₁-C₄ alkyl or C₁-C₄ haloalkyl;
Z is pyridinyl, pyrimidinyl, pyrazinyl, pyridazinyl or tetrahydrofuranyl, each unsubstituted or substituted with R¹⁹;
each R¹⁹ is independently halogen, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂—O₅ alkoxycarbonyl, C₂—O₅ alkylaminocarbonyl and C₃-C₅ dialkylaminocarbonyl;
R²⁰ is H, C₁-C₄ alkyl or C₁-C₄ haloalkyl;
R²¹ is fluoro, C₁-C₄ alkyl, C₁-C₄ haloalkyl, C₁-C₆ alkoxy, C₁-C₆ haloalkoxy, amino or C₁-C₆ alkylamino;
m is 0, 1 or 2; and
each n is independently 0, 1 or 2;
provided that
(i) when J is J-1, R¹ is Cl, R² is H and R³ is Cl, then R⁴ is other than —CH₂CH₃, —CH₂CF₃ or —CH₂(cyclopropyl);
(ii) when J is J-3, R¹ is Cl, R² is H, R³ is Cl and R⁷ is H, then Z is other than 2-pyridinyl; and
(iii) when J is J-5, R¹ is Cl, R² is H, R³ is Cl, and R¹⁰ and R¹¹ are H, then R¹² is other than —CH₂CF₃.
(iv) when R¹ is H, then R³ is other than H; and when R³ is H, then R¹ is other than H.

2. The Compound of claim 1 wherein R¹ is H, Cl or CF₃, R² is H or F, and R³ is Cl or CF₃.

3. The Compound of claim 1 or 2 wherein R¹ is H or Cl, R² is H or F, and R³ is Cl or CF₃.

4. The Compound of any one of claims 1 to 3 wherein R¹ is H or Cl, R² is H or F, and R³ is CF₃.

5. The Compound of any one of claims 1 to 4 wherein J is J-1, J-2 or J-3.

6. The Compound of any one of claims 1 to 5 wherein J is J-1.

7. The Compound of any one of claims 1 to 6 wherein R⁴ is C₁-C₆ alkyl, C₂-C₆ alkenyl, C₂-C₆ alkynyl, C₃-C₆ cycloalkyl, C₃-C₆ cycloalkenyl, C₄-C₈ alkylcycloalkyl.

8. The Compound of claim 7 wherein R⁴ is c-Pr or CH₂-c-Pr.

9. The Compound of claim 7 or 8 wherein R⁴ is substituted with substituents independently selected from halogen, cyano and CO₂R¹⁸.

10. The Compound of any one of claims 1 to 5 wherein J is J-3.

11. The Compound of any one of claims 1 to 5, and 10 wherein R⁷ is H or Me.

12. The compound of any one of claims 1 to 5, and 10 to 11 wherein Z is 2-pyridinyl or 2-pyrimidinyl.

13. The Compound of claim 1 wherein the Compound is at least one selected from 5-[5-(3,5-dichloro-4-fluorophenyl)-4,5-dihydro-5-(trifluoromethyl)-3-isoxazolyl]-N-(1,1-dimethylethyl)-8-isoquinolinecarboxamide (Compound 2), N-(cyclopropylmethyl)-5-[5-(3,5-dichloro-4-fluorophenyl)-4,5-dihydro-5-(trifluoromethyl)-3-isoxazolyl]-8-isoquinolinecarboxamide (Compound 3), 5-[5-(3,5-dichloro-4-fluorophenyl)-4,5-dihydro-5-(trifluoromethyl)-3-isoxazolyl]-N-(2-pyrimidinylmethyl)-8-isoquinolinecarboxamide (Compound 4), N-cyclopropyl-5-[5-(3,5-dichloro-4-fluorophenyl)-4,5-dihydro-5-(trifluoromethyl)-3-isoxazolyl]-8-isoquinolinecarboxamide (Compound 23), 5-[5-[3-chloro-5-(trifluoromethyl)phenyl]-4,5-dihydro-5-(trifluoromethyl)-3-isoxazolyl]-N-(cyclopropylmethyl)-8-isoquinolinecarboxamide (Compound 36), N-cyclopropyl-5-[4,5-dihydro-5-(trifluoromethyl)-5-[3-(trifluoromethyl)phenyl]-3-isoxazolyl]-8-isoquinolinecarboxamide (Compound 16), N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 17), N-(cyclopropylmethyl)-5-[5-[4-fluoro-3-(trifluoromethyl)phenyl]-4,5-dihydro-5-(trifluoromethyl)-3-isoxazolyl]-8-isoquinolinecarboxamide (Compound 58), 5-[5-[3-chloro-5-(trifluoromethyl)phenyl]-4,5-dihydro-5-(trifluoromethyl)-3-isoxazolyl]-N-cyclopropyl-8-isoquinolinecarboxamide (Compound 35), 5-[5-[4-fluoro-3-(trifluoromethyl)phenyl]-4,5-dihydro-5-(trifluoromethyl)-3-isoxazolyl]-N-2-propyn-1-yl-8-isoquinolinecarboxamide (Compound 74), 5-[5-(3,5-dichloro-4-fluorophenyl)-4,5-dihydro-5-(trifluoromethyl)-3-isoxazolyl]-N-ethyl-8-isoquinolinecarboxamide (Compound 46), 5-[5-(3,5-dichloro-4-fluorophenyl)-4,5-dihydro-5-(trifluoromethyl)-3-isoxazolyl]-N-2-propyn-1-yl-8-isoquinolinecarboxamide (Compound 48), N-cyclopropyl-5-[5-(3,5-dichlorophenyl)-4,5-dihydro-5-(trifluoromethyl)-3-isoxazolyl]-8-isoquinolinecarboxamide (Compound 70), N-cyclopropyl-5-[5-[4-fluoro-3-(trifluoromethyl)phenyl]-4,5-dihydro-5-(trifluoromethyl)-3-isoxazolyl]-8-isoquinolinecarboxamide (Compound 55), S—N-cyclopropyl-5-[5-(3,5-dichloro-4-fluorophenyl)-4,5-dihydro-5-(trifluoromethyl)-3-isoxazolyl]-8-isoquinolinecarboxamide (Compound 82), and S—N-(cyclopropylmethyl)-5-[4,5-dihydro-5-(trifluoromethyl)-5-[3-(trifluoromethyl)phenyl]-3-isoxazolyl]-8-isoquinolinecarboxamide (Compound 99).

14. A composition comprising a compound of any one of claims 1-13 and at least one additional component selected from the group consisting of surfactants, solid diluents and liquid diluents, said composition optionally further comprising at least one additional biologically active compound or agent.

15. The composition of claim 14 wherein the at least one additional biologically active compound or agent is selected from insecticides, fungicides, bactericides, nematocides, and herbicides.

16. The composition of claim 14 or 15 wherein the at least one additional biologically active compound or agent is selected from abamectin, acephate, acequinocyl, acetamiprid, acrinathrin, acynonapyr, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorantraniliprole, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cyantraniliprole (3-bromo-1-(3-chloro-2-pyridinyl)-N-[4-cyano-2-methyl-6-[(methyl amino)carbonyl]phenyl]-1H-pyrazole-5-carboxamide), cyclaniliprole (3-bromo-N-[2-bromo-4-chloro-O-[[(1-cyclopropyl ethyl)amino]carbonyl]phenyl]-1-(3-chloro-2-pyridinyl)-1H-pyrazole-5-carboxamide), cycloprothrin, cycloxaprid ((5S,8R)-1-[(6-chloro-3 pyridinyl)methyl]-2,3,5,6,7,8-hexahydro-9-nitro-5,8-Epoxy-1H-imidazo[1,2-a]azepine), cyenopyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromezotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, fluazaindolizine, flubendiamide, flucythrinate, flufenerim, flufenoxuron, flufenoxystrobin (methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-Rtrifluoromethyl)sulfinyl-1H-pyrazole-3-carbonitrile), flupyradifurone (4-[ [(6-chloro-3-pyridinyl)methyl](2,2-difluoroethyl)amino]-2(5H)-furanone), flupyrimin, fluvalinate, tau-fluvalinate, fluxametamide, fonophos, formetanate, fosthiazate, gamma-cyhalothrin, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, oxamyl, oxazosulfyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethypethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pydiflumetofen, pyriprole, pyriproxyfen, rotenone, ryanodine, silafluofen, spinetoram, spinosad, spirodiclofen, spiromesifen, spiropidion, spirotetramat, sulprofos, sulfoxaflor (N-[methyloxido[1-[6-(trifluoromethyl)-3-pyridinyl]ethyl]-sulfanylidene]cyanamide), tebufenozi de, tebufenpyrad, teflubenzuron, tefluthrin, kappa-tefluthrin, terbufos, tetrachlorantraniliprole, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, tyclopyrazoflor, zeta-cypermethrin, Bacillus thuringiensis delta-endotoxins, entomopathogenic bacteria, entomopathogenic viruses, and entomopathogenic fungi.

17. The composition of claim 3 wherein the at least one additional biologically active compound or agent is selected from cyantranilipcole, aretarniprid, imidacloprid, spirotetramat, chlorantraniliprole, bifenthrin, indoxacarb, avermectin, Bacillus spp. and any active crystal proteins, buprofezin, carbofuran, chlorfenapyr, chlorpyrifos, clothianidin, cyromazine, diafenthiuron, dinotefuran, emamectin benzoate, fipronil, flonicamid, fluhexafon, flupyradifurone, methomyl, methoxyfenozide, novaluron, permethrin, pyriproxifen, sulfoxaflor, thiamethoxam, γ-cyhalothrin, or -cypermethrin, broflanilide, dimpropyridaz, isocycloseram, tetrachlorantraniliprole, oxazosulfyl, tyclopyrazoflor, flupyrimin, spiropidion, acynonapyr, benzpyrimoxan, chloroprallethrin, epsilon-metofluthrin, kappa-bifenthrin, dicloromezotiaz, and kappa-tefluthrin.

18. A liquid or dry formulation comprising the compound or composition of any one of claims 1-17 for use in a drip irrigation system, furrow during planting, handheld sprayer, backpack sprayer, boom sprayer, ground sprayer, aerial application, unmanned aerial vehicle, or a seed treatment.

19. A method for controlling an invertebrate pest comprising contacting the invertebrate pest or its environment with a biologically effective amount of a compound, composition or formulation of any one of claims 1-18.

20. A treated seed comprising a compound of claim 1 in an amount of from about 0.0001 to 1% by weight of the seed before treatment.
